SEQUENCE LISTING (24 sequences)

SEQ ID NO: 1 - protein, 248 aa, Homo sapiens
MSLFPSLPLLLLSMVAASYSETVTCEDAQKTCPAVIACSSPGINGFPGKDGRDGTKGEKGEPGQGLRGLQGPPGKLGPPGNPGPSGSPGPKGQKGDPGKSPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 2 - protein, 228 aa, Homo sapiens
ETVTCEDAQKTCPAVIACSSPGINGFPGKDGRDGTKGEKGEPGQGLRGLQGPPGKLGPPGNPGPSGSPGPKGQKGDPGKSPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 3 - protein, 141 aa, Homo sapiens
AASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 4 - protein, 115 aa, Homo sapiens
VGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 5 - protein, 148 aa, Homo sapiens
PDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 6 - protein, 381 aa, Artificial Sequence
  Recombinantly-produced fusion protein
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAPDGDSSLAASERKALQTEMARIKKWLTFSLGKQVGNKFFLTNGEINITFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 7 - protein, 383 aa, Artificial Sequence
  Recombinantly-produced fusion protein
AKTEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 8 - protein, 352 aa, Artificial Sequence
  Recombinantly-produced fusion protein
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGATSKQVGNKFFLTNGEEVITFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 9 - protein, 232 aa, Homo sapiens
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGA

SEQ ID NO: 10 - protein, 10 aa, Homo sapiens
NEGEPNNAGS

SEQ ID NO: 11 - protein, 15 aa, Homo sapiens
LLLKNGQWNDVPCST

SEQ ID NO: 12 - protein, 326 aa, Homo sapiens
MELSGATMARGLAVLLVLFLHIKNLPAQAADTCPEVKVVGLEGSDKLTILRGCPGLPGAPGPKGEAGVIGERGERGLPGAPGKAGPVGPKGDRGEKGMRGEKGDAGQSQSCATGPRNCKDLLDRGYFLSGWHTIYLPDCRPLTVLCDMDTDGGGWTVFQRRMDGSVDFYRDWAAYKQGFGSQLGEFWLGNDNIHALTAQGSSELRVDLVDFEGNHQFAKYKSFKVADEAEKYKLVLGAFVGGSAGNSLTGHNNNFFSTKDQDNDVSSSNCAEKFQGAWWYADCHASNLNGLYLMGPHESYANGINWSAAKGYKYSYKVSEMKVRPA

SEQ ID NO: 13 - protein, 313 aa, Homo sapiens
MELDRAVGVLGAATLLLSFLGMAWALQAADTCPEVKMVGLEGSDKLTILRGCPGLPGAPGPKGEAGTNGKRGERGPPGPPGKAGPPGPNGAPGEPQPCLTGPRTCKDLLDRGHFLSGWHTIYLPDCRPLTVLCDMDTDGGGWTVFQRRVDGSVDFYRDWATYKQGFGSRLGEFWLGNDNIHALTAQGTSELRVDLVDFEDNYQFAKYRSFKVADEAEKYNLVLGAFVEGSAGDSLTFHNNQSFSTKDQDNDLNTGNCAVMFQGAWWYKNCHVSNLNGRYLRGTHGSFANGINWKSGKGYNYSYKVSEMKVRPA

SEQ ID NO: 14 - protein, 299 aa, Homo sapiens
MDLLWILPSLWLLLLGGPACLKTQEHPSCPGPRELEASKVVLLPSCPGAPGSPGEKGAPGPQGPPGPPGKMGPKGEPGDPVNLLRCQEGPRNCRELLSQGATLSGWYHLCLPEGRALPVFCDMDTEGGGWLVFQRRQDGSVDFFRSWSSYRAGFGNQESEFWLGNENLHQLTLQGNWELRVELEDFNGNRTFAHYATFRLLGEVDHYQLALGKFSEGTAGDSLSLHSGRPFTTYDADHDSSNSNCAVIVHGAWWYASCYRSNLNGRYAVSEAAAHKYGIDWASGRGVGHPYRRVRMMLR

SEQ ID NO: 15 - protein, 786 aa, Homo sapiens
MTSIFHFAIIFMLILQIRIQLSEESEFLVDRSKNGLIHVPKDLSQKTTILNISQNYISELWTSDILSLSKLRILIISHNRIQYLDISVFKFNQELEYLDLSHNKLVKISCHPTVNLKHLDLSFNAFDALPICKEFGNMSQLKFLGLSTTHLEKSSVLPIAHLNISKVLLVLGETYGEKEDPEGLQDFNTESLHIVFPTNKEFHFILDVSVKTVANLELSNIKCVLEDNKCSYFLSILAKLQTNPKLSNLTLNNIETTWNSFIRILQLVWHTTVWYFSISNVKLQGQLDFRDFDYSGTSLKALSIHQVVSDVFGFPQSYIYEIFSNMNIKNFTVSGTRMVHMLCPSKISPFLHLDFSNNLLTDTVFENCGHLTELETLILQMNQLKELSKIAEMTTQMKSLQQLDISQNSVSYDEKKGDCSWTKSLLSLNMSSNILTDTIFRCLPPRIKVLDLHSNKIKSIPKQVVKLEALQELNVAFNSLTDLPGCGSFSSLSVLIIDHNSVSHPSADFFQSCQKMRSIKAGDNPFQCTCELGEFVKNIDQVSSEVLEGWPDSYKCDYPESYRGTLLKDFHMSELSCNITLLIVTIVATMLVLAVTVTSLCSYLDLPWYLRMVCQWTQTRRRARNIPLEELQRNLQFHAFISYSGHDSFWVKNELLPNLEKEGMQICLHERNFVPGKSIVENIITCIEKSYKSIFVLSPNFVQSEWCHYELYFAHHNLFHEGSNSLILILLEPIPQYSIPSSYHKLKSLMARRTYLEWPKEKSKRGLFWANLRAAINIKLTEQAKK

SEQ ID NO: 16 - protein, 784 aa, Homo sapiens
MPHTLWMVWVLGVIISLSKEESSNQASLSCDRNGICKGSSGSLNSIPSGLTEAVKSLDLSNNRITYISNSDLQRCVNLQALVLTSNGINTIEEDSFSSLGSLEHLDLSYNYLSNLSSSWFKPLSSLTFLNLLGNPYKTLGETSLFSHLTKLQILRVGNMDTFTKIQRKDFAGLTFLEELEIDASDLQSYEPKSLKSIQNVSHLILHMKQHILLLEIFVDVTSSVECLELRDTDLDTFHFSELSTGETNSLIKKFTFRNVKITDESLFQVMKLLNQISGLLELEFDDCTLNGVGNFRASDNDRVIDPGKVETLTIRRLHIPRFYLFYDLSTLYSLTERVKRITVENSKVFLVPCLLSQHLKSLEYLDLSENLMVEEYLKNSACEDAWPSLQTLILRQNHLASLEKTGETLLTLKNLTNIDISKNSFHSMPETCQWPEKMKYLNLSSTRIHSVTGCIPKTLEILDVSNNNLNLFSLNLPQLKELYISRNKLMTLPDASLLPMLLVLKISRNAITTFSKEQLDSFHTLKTLEAGGNNFICSCEFLSFTQEQQALAKVLIDWPANYLCDSPSHVRGQQVQDVRLSVSECHRTALVSGMCCALFLLILLTGVLCHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKS

SEQ ID NO: 17 - protein, 904 aa, Homo sapiens
MRQTLPCIYFWGGLLPFGMLCASSTTKCTVSHEVADCSHLKLTQVPDDLPTNITVLNLTHNQLRRLPAANFTRYSQLTSLDVGFNTISKLEPELCQKLPMLKVLNLQHNELSQLSDKTFAFCTNLTELHLMSNSIQKIKNNPFVKQKNLITLDLSHNGLSSTKLGTQVQLENLQELLLSNNKIQALKSEELDIFANSSLKKLELSSNQIKEFSPGCFHAIGRLFGLFLNNVQLGPSLTEKLCLELANTSIRNLSLSNSQLSTTSNTTFLGLKWTNLTMLDLSYNNLNVVGNDSFAWLPQLEYFFLEYNNIQHLFSHSLHGLFNVRYLNLKRSFTKQSISLASLPKIDDFSFQWLKCLEHLNMEDNDIPGIKSNMFTGLINLKYLSLSNSFTSLRTLTNETFVSLAHSPLHILNLTKNKISKIESDAFSWLGHLEVLDLGLNEIGQELTGQEWRGLENIFEIYLSYNKYLQLTRNSFALVPSLQRLMLRRVALKNVDSSPSPFQPLRNLTILDLSNNNIANINDDMLEGLEKLEILDLQHNNLARLWKHANPGGPIYFLKGLSHLHILNLESNGFDEIPVEVFKDLFELKIIDLGLNNLNTLPASVFNNQVSLKSLNLQKNLITSVEKKVFGPAFRNLTELDMRFNPFDCTCESIAWFVNWINETHTNIPELSSHYLCNTPPHYHGFPVRLFDTSSCKDSAPFELFFMINTSILLIFIFIVLLIHFEGWRISFYWNVSVHRVLGFKEIDRQTEQFEYAAYIIHAYKDKDWVWEHFSSMEKEDQSLKFCLEERDFEAGVFELEAIVNSIKRSRKIIFVITHHLLKDPLCKRFKVHHAVQQAIEQNLDSIILVFLEEIPDYKLNHALCLRRGMFKSHCILNWPVQKERIGAFRHKLQVALGSKNSVH

SEQ ID NO: 18 - protein, 839 aa, Homo sapiens
MMSASRLAGTLIPAMAFLSCVRPESWEPCVEVVPNITYQCMELNFYKIPDNLPFSTKNLDLSFNPLRHLGSYSFFSFPELQVLDLSRCEIQTIEDGAYQSLSHLSTLILTGNPIQSLALGAFSGLSSLQKLVAVETNLASLENFPIGHLKTLKELNVAHNLIQSFKLPEYFSNLTNLEHLDLSSNKIQSIYCTDLRVLHQMPLLNLSLDLSLNPMNFIQPGAFKEIRLHKLTLRNNFDSLNVMKTCIQGLAGLEVHRLVLGEFRNEGNLEKFDKSALEGLCNLTIEEFRLAYLDYYLDDIIDLFNCLTNVSSFSLVSVTIERVKDFSYNFGWQHLELVNCKFGQFPTLKLKSLKRLTFTSNKGGNAFSEVDLPSLEFLDLSRNGLSFKGCCSQSDFGTTSLKYLDLSFNGVITMSSNFLGLEQLEHLDFQHSNLKQMSEFSVFLSLRNLIYLDISHTHTRVAFNGIFNGLSSLEVLKMAGNSFQENFLPDIFTELRNLTFLDLSQCQLEQLSPTAFNSLSSLQVLNMSHNNFFSLDTFPYKCLNSLQVLDYSLNHIMTSKKQELQHFPSSLAFLNLTQNDFACTCEHQSFLQWIKDQRQLLVEVERMECATPSDKQGMPVLSLNITCQMNKTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 19 - protein, 858 aa, Homo sapiens
MGDHLDLLLGVVLMAGPVFGIPSCSFDGRIAFYRFCNLTQVPQVLNTTERLLLSFNYIRTVTASSFPFLEQLQLLELGSQYTPLTIDKEAFRNLPNLRILDLGSSKIYFLHPDAFQGLFHLFELRLYFCGLSDAVLKDGYFRNLKALTRLDLSKNQIRSLYLHPSFGKLNSLKSIDFSSNQIFLVCEHELEPLQGKTLSFFSLAANSLYSRVSVDWGKCMNPFRNMVLEILDVSGNGWTVDITGNFSNAISKSQAFSLILAHHIMGAGFGFHNIKDPDQNTFAGLARSSVRHLDLSHGFVFSLNSRVFETLKDLKVLNLAYNKINKIADEAFYGLDNLQVLNLSYNLLGELYSSNFYGLPKVAYIDLQKNHIAIIQDQTFKFLEKLQTLDLRDNALTTIHFIPSIPDIFLSGNKLVTLPKINLTANLIHLSENRLENLDILYFLLRVPHLQILILNQNRFSSCSGDQTPSENPSLEQLFLGENMLQLAWETELCWDVFEGLSHLQVLYLNHNYLNSLPPGVFSHLTALRGLSLNSNRLTVLSHNDLPANLEILDISRNQLLAPNPDVFVSLSVLDITHNKFICECELSTFINWLNHTNVTIAGPPADIYCVYPDSFSGVSLFSLSTEGCDEEEVLKSLKFSLFIVCTVTLTLFLMTILTVTKFRGFCFICYKTAQRLVFKDHPQGTEPDMYKYDAYLCFSSKDFTWVQNALLKHLDTQYSDQNRFNLCFEERDFVPGENRIANIQDAIWNSRKIVCLVSRHFLRDGWCLEAFSYAQGRCLSDLNSALIMVVVGSLSQYQLMKHQSIRGFVQKQQYLRWPEDLQDVGWFLHKLSQQILKKEKEKKKDNNIPLQTVATIS

SEQ ID NO: 20 - protein, 796 aa, Homo sapiens
MTKDKEPIVKSFHFVCLMIIIVGTRIQFSDGNEFAVDKSKRGLIHVPKDLPLKTKVLDMSQNYIAELQVSDMSFLSELTVLRLSHNRIQLLDLSVFKFNQDLEYLDLSHNQLQKISCHPIVSFRHLDLSFNDFKALPICKEFGNLSQLNFLGLSAMKLQKLDLLPIAHLHLSYILLDLRNYYIKENETESLQILNAKTLHLVFHPTSLFAIQVNISVNTLGCLQLTNIKLNDDNCQVFIKFLSELTRGSTLLNFTLNHIETTWKCLVRVFQFLWPKPVEYLNIYNLTIIESIREEDFTYSKTTLKALTIEHITNQVFLFSQTALYTVFSEMNIMMLTISDTPFIHMLCPHAPSTFKFLNFTQNVFTDSIFEKCSTLVKLETLILQKNGLKDLFKVGLMTKDMPSLEILDVSWNSLESGRHKENCTWVESIVVLNLSSNMLTDSVFRCLPPRIKVLDLHSNKIKSVPKQVVKLEALQELNVAFNSLTDLPGCGSFSSLSVLIIDHNSVSHPSADFFQSCQKMRSIKAGDNPFQCTCELREFVKNIDQVSSEVLEGWPDSYKCDYPESYRGSPLKDFHMSELSCNITLLIVTIGATMLVLAVTVTSLCIYLDLPWYLRMVCQWTQTRRRARNIPLEELQRNLQFHAFISYSEHDSAWVKSELVPYLEKEDIQICLHERNFVPGKSIVENIINCIEKSYKSIFVLSPNFVQSEWCHYELYFAHHNLFHEGSNNLILILLEPIPQNSIPNKYHKLKALMTQRTYLQWPKEKSKRGLFWANIRAAFNMKLTLVTENNDVKS

SEQ ID NO: 21 - protein, 1049 aa, Homo sapiens
MVFPMWTLKRQILILFNIILISKLLGARWFPKTLPCDVTLDVPKNHVIVDCTDKHLTEIPGGIPTNTTNLTLTINHIPDISPASFHRLDHLVEIDFRCNCVPIPLGSKNNMCIKRLQIKPRSFSGLTYLKSLYLDGNQLLEIPQGLPPSLQLLSLEANNIFSIRKENLTELANIEILYLGQNCYYRNPCYVSYSIEKDAFLNLTKLKVLSLKDNNVTAVPTVLPSTLTELYLYNNMIAKIQEDDFNNLNQLQILDLSGNCPRCYNAPFPCAPCKNNSPLQIPVNAFDALTELKVLRLHSNSLQHVPPRWFKNINKLQELDLSQNFLAKEIGDAKFLHFLPSLIQLDLSFNFELQVYRASMNLSQAFSSLKSLKILRIRGYVFKELKSFNLSPLHNLQNLEVLDLGTNFIKIANLSMFKQFKRLKVIDLSVNKISPSGDSSEVGFCSNARTSVESYEPQVLEQLHYFRYDKYARSCRFKNKEASFMSVNESCYKYGQTLDLSKNSIFFVKSSDFQHLSFLKCLNLSGNLISQTLNGSEFQPLAELRYLDFSNNRLDLLHSTAFEELHKLEVLDISSNSHYFQSEGITHMLNFTKNLKVLQKLMMNDNDISSSTSRTMESESLRTLEFRGNHLDVLWREGDNRYLQLFKNLLKLEELDISKNSLSFLPSGVFDGMPPNLKNLSLAKNGLKSFSWKKLQCLKNLETLDLSHNQLTTVPERLSNCSRSLKNLILKNNQIRSLTKYFLQDAFQLRYLDLSSNKIQMIQKTSFPENVLNNLKMLLLHHNRFLCTCDAVWFVWWVNHTEVTIPYLATDVTCVGPGAHKGQSVISLDLYTCELDLTNLILFSLSISVSLFLMVMMTASHLYFWDVWYIYHFCKAKIKGYQRLISPDCCYDAFIVYDTKDPAVTEWVLAELVAKLEDPREKHFNLCLEERDWLPGQPVLENLSQSIQLSKKTVFVMTDKYAKTENFKIAFYLSHQRLMDEKVDVIILIFLEKPFQKSKFLQLRKRLCGSSVLEWPTNPQAHPYFWQCLKNALATDNHVAYSQVFKETV

SEQ ID NO: 22 - protein, 1041 aa, Homo sapiens
MENMFLQSSMLTCIFLLISGSCELCAEENFSRSYPCDEKKQNDSVIAECSNRRLQEVPQTVGKYVTELDLSDNFITHITNESFQGLQNLTKINLNHNPNVQHQNGNPGIQSNGLNITDGAFLNLKNLRELLLEDNQLPQIPSGLPESLTELSLIQNNIYNITKEGISRLINLKNLYLAWNCYFNKVCEKTNIEDGVFETLTNLELLSLSFNSLSHVPPKLPSSLRKLFLSNTQIKYISEEDFKGLINLTLLDLSGNCPRCFNAPFPCVPCDGGASINIDRFAFQNLTQLRYLNLSSTSLRKINAAWFKNMPHLKVLDLEFNYLVGEIASGAFLTMLPRLEILDLSFNYIKGSYPQHINISRNFSKLLSLRALHLRGYVFQELREDDFQPLMQLPNLSTINLGINFIKQIDFKLFQNFSNLEIIYLSENRISPLVKDTRQSYANSSSFQRHIRKRRSTDFEFDPHSNFYHFTRPLIKPQCAAYGKALDLSLNSIFFIGPNQFENLPDIACLNLSANSNAQVLSGTEFSAIPHVKYLDLTNNRLDFDNASALTELSDLEVLDLSYNSHYFRIAGVTHHLEFIQNFTNLKVLNLSHNNIYTLTDKYNLESKSLVELVFSGNRLDILWNDDDNRYISIFKGLKNLTRLDLSLNRLKHIPNEAFLNLPASLTELHINDNMLKFFNWTLLQQFPRLELLDLRGNKLLFLTDSLSDFTSSLRTLLLSHNRISHLPSGFLSEVSSLKHLDLSSNLLKTINKSALETKTTTKLSMLELHGNPFECTCDIGDFRRWMDEHLNVKIPRLVDVICASPGDQRGKSIVSLELTTCVSDVTAVILFFFTFFITTMVMLAALAHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 23 - protein, 1032 aa, Homo sapiens
MGFCRSALHPLSLLVQAIMLAMTLALGTLPAFLPCELQPHGLVNCNWLFLKSVPHFSMAAPRGNVTSLSLSSNRIHHLHDSDFAHLPSLRHLNLKWNCPPVGLSPMHFPCHMTIEPSTFLAVPTLEELNLSYNNIMTVPALPKSLISLSLSHTNILMLDSASLAGLHALRFLFMDGNCYYKNPCRQALEVAPGALLGLGNLTHLSLKYNNLTVVPRNLPSSLEYLLLSYNRIVKLAPEDLANLTALRVLDVGGNCRRCDHAPNPCMECPRHFPQLHPDTFSHLSRLEGLVLKDSSLSWLNASWFRGLGNLRVLDLSENFLYKCITKTKAFQGLTQLRKLNLSFNYQKRVSFAHLSLAPSFGSLVALKELDMHGIFFRSLDETTLRPLARLPMLQTLRLQMNFINQAQLGIFRAFPGLRYVDLSDNRISGASELTATMGEADGGEKVWLQPGDLAPAPVDTPSSEDFRPNCSTLNFTLDLSRNNLVTVQPEMFAQLSHLQCLRLSHNCISQAVNGSQFLPLTGLQVLDLSHNKLDLYHEHSFTELPRLEALDLSYNSQPFGMQGVGHNFSFVAHLRTLRHLSLAHNNIHSQVSQQLCSTSLRALDFSGNALGHMWAEGDLYLHFFQGLSGLIWLDLSQNRLHTLLPQTLRNLPKSLQVLRLRDNYLAFFKWWSLHFLPKLEVLDLAGNQLKALTNGSLPAGTRLRRLDVSCNSISFVAPGFFSKAKELRELNLSANALKTVDHSWFGPLASALQILDVSANPLHCACGAAFMDFLLEVQAAVPGLPSRVKCGSPGQLQGLSIFAQDLRLCLDEALSWDCFALSLLAVALGLGVPMLHHLCGWDLWYCFHLCLAWLPWRGRQSGRDEDALPYDAFVVFDKTQSAVADWVYNELRGQLEECRGRWALRLCLEERDWLPGKTLFENLWASVYGSRKTLFVLAHTDRVSGLLRASFLLAQQRLLEDRKDVVVLVILSPDGRRSRYVRLRQRLCRQSVLLWPHQPSGQRSFWAQLGMALTRDNHHFYNRNFCQGPTAE

SEQ ID NO: 24 - protein, 811 aa, Homo sapiens
MRLIRNIYIFCSIVMTAEGDAPELPEERELMTNCSNMSLRKVPADLTPATTTLDLSYNLLFQLQSSDFHSVSKLRVLILCHNRIQQLDLKTFEFNKELRYLDLSNNRLKSVTWYLLAGLRYLDLSFNDFDTMPICEEAGNMSHLEILGLSGAKIQKSDFQKIAHLHLNTVFLGFRTLPHYEEGSLPILNTTKLHIVLPMDTNFWVLLRDGIKTSKILEMTNIDGKSQFVSYEMQRNLSLENAKTSVLLLNKVDLLWDDLFLILQFVWHTSVEHFQIRNVTFGGKAYLDHNSFDYSNTVMRTIKLEHVHFRVFYIQQDKIYLLLTKMDIENLTISNAQMPHMLFPNYPTKFQYLNFANNILTDELFKRTIQLPHLKTLILNGNKLETLSLVSCFANNTPLEHLDLSQNLLQHKNDENCSWPETVVNMNLSYNKLSDSVFRCLPKSIQILDLNNNQIQTVPKETIHLMALRELNIAFNFLTDLPGCSHFSRLSVLNIEMNFILSPSLDFVQSCQEVKTLNAGRNPFRCTCELKNFIQLETYSEVMMVGWSDSYTCEYPLNLRGTRLKDVHLHELSCNTALLIVTIVVIMLVLGLAVAFCCLHFDLPWYLRMLGQCTQTWHRVRKTTQEQLKRNVRFHAFISYSEHDSLWVKNELIPNLEKEDGSILICLYESYFDPGKSISENIVSFIEKSYKSIFVLSPNFVQNEWCHYEFYFAHHNLFHENSDHIILILLEPIPFYCIPTRYHKLKALLEKKAYLEWPKDRRKCGLFWANLRAAINVNVLATREMYELQTFTELNEESRGSTISLMRTDCL

(19) United States
(12) Patent Application Publication
(10) Pub. No.: US 2024/0011980 A1
(43) Pub. Date: Jan. 11, 2024

(54) LATERAL FLOW ASSAY FOR RAPID DETECTION OF PATHOGENS IN SAMPLES

(71) Applicant: MIRAKI INNOVATION THINK TANK LLC, Cambridge, MA (US)

(72) Inventors: Nisha Veronica VARMA, Brookline, MA (US); Goossen Jan Bernard BOER, Brookline, MA (US); Zhiqian ZHOU, Boston, MA (US); George A. DOWNEY, Arlington, MA (US); Shailendra KUMAR, Needham, MA (US); Keith CRAWFORD, Westwood, MA (US)

(73) Assignee: MIRAKI INNOVATION THINK TANK LLC, Cambridge, MA (US)

(21) Appl. No.: 18/033,221

(22) PCT Filed: Jun. 11, 2021
(86) PCT No.: PCT/US2021/037021

§ 371 Date: Apr. 21, 2023

Related U.S. Application Data
(60) Provisional application No. 63/094,619, filed on Oct. 21, 2020.
(60) Provisional application No. 63/038,239, filed on Jun. 12, 2020.

(51) Int. Cl. G01N 33/543; G01N 33/569

(52) U.S. Cl. CPC G01N 33/54388; G01N 33/56916; G01N 33/56938; G01N 33/56961; G01N 2333/245; G01N 2333/395

(57) ABSTRACT

Detection devices comprising microbe-targeting molecules (MTMs) and engineered MTMs in the form of a lateral flow assay (LFA) are provided. Methods of using the detection devices in the detection and/or identification of microbes and microbe components in a sample are also provided.

### SEQUENCE LISTING

A sequence listing in electronic (ASCII text file) format is filed with this application and incorporated herein by reference. The name of the ASCII text file is “2021_0917A_ST25.txt”; the file was created on Jun. 9, 2021; the size of the file is 103 KB.

### FIELD OF THE INVENTION

The technology described herein generally relates to detection devices comprising unique biological molecules that bind to microbes and microbial components in samples (e.g. bodily and environmental fluids), and to methods of using such devices in detecting analyte in a sample.

### BACKGROUND

There is an increasing need in the field of health care for affordable and highly sensitive tools that can be used to rapidly and accurately identify the presence of microbes and microbial components in a sample, such as a biological or environmental sample. Such tools can be incorporated into detection devices for use in screening samples for the presence of selected organisms. Important microbes and their components include, for example, environmental and pathogenic agents, including bacteria, viruses, toxins, and antigens. To meet this need, a variety of biosensor products have been commercially developed and released.

A specific example of a biosensor platform currently in use is the CANARY® biosensor technology of PathSensors, Inc. This platform, based on the work of Rider et al. (Science 2003, 301:213-215), enables reliable identification of specific airborne and liquid-based pathogens. The biological backbone of the CANARY® biosensor is comprised of a genetically-engineered B cell expressing an extracellularly bound, antigen-specific antibody that can bind its cognate antigen or pathogenic agent. In this system, when an antigen-containing sample interacts with the antibody on the extracellular surface of the biosensor, an intracellular signaling cascade is activated resulting in the release of Ca²⁺ within the B cells. In the CANARY® system, the B cells express aequorin, a Ca²⁺-sensitive photoprotein, which results in cell luminescence in the presence of elevated intracellular Ca²⁺ levels. Thus, the luminescence can be used to indicate antigen binding.

The CANARY® system can be used to efficiently identify a number of specific antigens, including those from bacteria, viruses, and toxins. However, expansion of the antigen test repertoire is complex and costly. Different antigen- or pathogen-specific biosensors must be constructed to recognized each and every selected antigen, which requires multiple steps including production of hybridoma cell lines, cloning of nucleic acid sequences encoding the antibodies, and expressing cloned antibodies as transmembrane proteins on the surface of a B cell line genetically engineered to luminesce upon binding of the cognate antigen (e.g., a pathogen) by the antibody. Use of the system to detect more than a handful of different organisms remains limited.

Thus, there remains a need for the development of a universal and near-universal biosensor tool that can be adapted for use in multiple detection platforms across a broad range of environmental and pathogenic agents, and that can also be incorporated into detection devices. The present invention is directed to this and other important goals.

### BRIEF SUMMARY

The present invention is generally directed to detection devices and methods of using such devices, where the devices comprise microbe-targeting molecules (MTMs) and/or engineered MTMs that have the shared characteristic of binding to one or more microbe-associated molecular patterns (MAMPs).

The detection devices of the invention can be used in the detection and/or identification of microbes in a sample, such as a biological or environmental sample.

In one aspect, the detection devices of the invention are in the form of lateral flow assays (LFA) or vertical flow assays (LFA), and the methods of the invention are directed to the use of such devices in the detection selected analytes in a sample.

The important basis of the MTMs used in the detection devices of the invention, and the related methods, is that these constructs contact and bind microbes and microbial components in a sample based on the identity of the MAMP produced by the microbe, rather than the identity of microbe itself. While some MAMPs are produced by only a single species of microbe, other MAMPs are shared across species. Thus, while some MTMs of the invention bind to only MAMPs of a particular species of microbe, other MTMs of the invention can bind to MAMPs produced by all members of a particular class, order, family, genus or sub-genus of microbe.

### MTMs

Before discussing the physical structure of the detection devices of the invention, and the related methods, it will be helpful to summarize the characteristics of the microbe-targeting molecules (MTMs). As used herein, “MTM” and “engineered MTM” refers to any of the molecules described herein (or described in patents or patent application incorporated by reference) that can bind to a microbe or microbe component. Unless the context indicates otherwise, the term “MTM” is used to describe all MTMs of the invention, both naturally-occurring and engineered forms of these constructs.

Given that the MTMs of the invention are defined based on their binding activity, it will be apparent that both naturally-occurring and engineered MTMs will comprise at least one microbe-binding domain, i.e. a domain that recognizes and binds to one or more MAMPs (including, at least two, at least three, at least four, at least five, or more) as described herein. A microbe-binding domain can be a naturally-occurring or a synthetic molecule. In some aspects, a microbe-binding domain can be a recombinant molecule. In addition to the microbe-binding domain, the MTMs of the invention will typically have one or more additional domains that may include, but are not limited to, an oligomerization domain, a signal domain, an anchor domain, a collagen-like domain, a fibrinogen-like domain, an immunoglobulin domain, and an immunoglobulin-like domain.

As non-limiting examples of the MTMs of the invention, three broad categories of suitable MTMs are encompassed within the invention, namely: (i) collectin-based MTMs, (ii) ficolin-based MTMs, and (iii) toll-like receptor-based MTMs.

Thus, and in a first embodiment, the present invention is directed detection devices comprising collectin-based engineered MTMs. These collectin-based engineered MTMs comprise at least one collectin microbe-binding domain and at least one additional domain.

The collectin microbe-binding domain comprises a carbohydrate recognition domain (CRD) of a collectin. The collectin may be any one of (i) mannose-binding lectin (MBL), (ii) surfactant protein A (SP-A), (iii) surfactant protein D (SP-D), (iv) collectin liver 1 (CL-L1), (v) collectin placenta 1 (CL-P1), (vi) conglutinin collectin of 43 kDa (CL-43), (vii) collectin of 46 kDa (CL-46), (viii) collectin kidney 1 (CL-K1), (ix) conglutinin, and (x) a sequence variant having at least 85% sequence identity to any one of (i)-(ix).

The at least one additional domain may be one or more of (xi) a collectin cysteine-rich domain, (xii) a collectin collagen-like domain, (xiii) a collectin coiled-coil neck domain, (xiv) a ficolin short N-terminal domain, (xv) a ficolin collagen-like domain, (xvi) a TLR transmembrane helix, (xvii) a TLR C-terminal cytoplasmic signaling domain, (xviii) an oligomerization domain, (xix) a signal domain, (xx) an anchor domain, (xxi) a collagen-like domain, (xxii) a fibrinogen-like domain, (xxiii) an immunoglobulin domain, (xxiv) an immunoglobulin-like domain, and (xxv) a sequence variant having at least 85% sequence identity to any one of (xi)-(xxiv).

In certain aspects of this embodiment, the at least one additional domain is an immunoglobulin domain. For example, the immunoglobulin domain may comprise the amino acid sequence of SEQ ID NO:9 or a sequence variant thereof having at least 85% sequence identity to SEQ ID NO:9.

In certain aspects of this embodiment, the collectin microbe-binding domain comprises the CRD of MBL or a sequence variant thereof having at least 85% sequence identity to the CRD of MBL. For example, the CRD of MBL may comprise the amino acid sequence of any one of SEQ ID NOs:1, 2, 3, 4, and 5 or a sequence variant thereof having at least 85% sequence identity to any one of SEQ ID NOs:1, 2, 3, 4, and 5.

In certain aspects of this embodiment, the collectin microbe-binding domain comprises the CRD of MBL or a sequence variant thereof having at least 85% sequence identity to the CRD of MBL and an immunoglobulin domain. For example, the CRD of MBL may comprise the amino acid sequence of any one of SEQ ID NOs:1, 2, 3, 4, and 5 or a sequence variant thereof having at least 85% sequence identity to any one of SEQ ID NOs:1, 2, 3, 4, and 5, and an immunoglobulin domain comprising the amino acid sequence of SEQ ID NO:9 or a sequence variant thereof having at least 85% sequence identity to SEQ ID NO:9.

In certain aspects of this embodiment, the detection devices comprise at least one collectin-based engineered MTM, wherein the collectin-based engineered MTM is an FcMBL of SEQ ID NO:6, 7 or 8 or a sequence variant thereof having at least 85% sequence identity to SEQ ID NO:6, 7 or 8. As indicated above, FcMBL MTMs comprise a mannose-binding ligand (MBL) linked to the Fc domain of human IgG (Fc).

In certain other aspects of this embodiment, the detection devices comprise at least two collectin-based engineered MTMs, wherein the two MTMs are selected from SEQ ID NO:6, 7 or 8 or a sequence variant thereof having at least 85% sequence identity to SEQ ID NO:6, 7 or 8.

In a second embodiment, the invention is directed to detection devices comprising ficolin-based engineered MTMs. These ficolin-based engineered MTMs comprise at least one ficolin microbe-binding domain and at least one additional domain.

The ficolin microbe-binding domain may comprise the fibrinogen-like domain of a ficolin. The ficolin may be any one of (i) ficolin 1, (ii) ficolin 2, (iii) ficolin 3, and (iv) a sequence variant having at least 85% sequence identity to any one of (i)-(iii).

The at least one additional domain may be one or more of (v) a ficolin short N-terminal domain, (vi) a ficolin collagen-like domain, (vii) a collectin cysteine-rich domain, (viii) a collectin collagen-like domain, (ix) a collectin coiled-coil neck domain, (x) a TLR transmembrane helix, (xi) a TLR C-terminal cytoplasmic signaling domain, (xii) an oligomerization domain, (xiii) a signal domain, (xiv) an anchor domain, (xv) a collagen-like domain, (xvi) a fibrinogen-like domain, (xvii) an immunoglobulin domain, (xviii) an immunoglobulin-like domain, and (xix) a sequence variant having at least 85% sequence identity to any one of (v)-(xviii).

In certain aspects of this embodiment, the at least one additional domain is an immunoglobulin domain. For example, the immunoglobulin domain may comprise the amino acid sequence of SEQ ID NO:9 or a sequence variant thereof having at least 85% sequence identity to SEQ ID NO:9.

In certain aspects of this embodiment, the ficolin-based engineered MTMs comprise a ficolin microbe-binding domain comprising the fibrinogen-like domain of any one of SEQ ID NOs:12, 13 and 14 or a sequence variant thereof having at least 85% sequence identity to any one of SEQ ID NOs:12, 13 and 14.

In certain aspects of this embodiment, the ficolin-based engineered MTMs comprise a ficolin microbe-binding domain comprising the fibrinogen-like domain of any one of SEQ ID NOs:12, 13 and 14 or a sequence variant thereof having at least 85% sequence identity to any one of SEQ ID NOs:12, 13 and 14 and an immunoglobulin domain comprising the amino acid sequence of SEQ ID NO:9 or a sequence variant thereof having at least 85% sequence identity to SEQ ID NO:9.

In a third embodiment, the invention is directed to detection devices comprising toll-like receptor (TLR)-based engineered MTMs. These TLR-based engineered MTMs comprise at least one TLR microbe-binding domain and at least one additional domain.

The TLR microbe-binding domain may comprise the N-terminal ligand-binding domain of a TLR. The TLR may be any one of (i) TLR1, (ii) TLR2, (iii) TLR3, (iv) TLR4, (v) TLR5, (vi) TLR6, (vii) TLR7, (viii) TLR8, (ix) TLR9, (x) TLR10, and (xi) a sequence variant having at least 85% sequence identity to any one of (i)-(x).

The at least one additional domain may be one or more of (xii) a TLR transmembrane helix, (xiii) a TLR C-terminal cytoplasmic signaling domain, (xiv) a ficolin short N-terminal domain, (xv) a ficolin collagen-like domain, (xvi) a collectin cysteine-rich domain, (xvii) a collectin collagen-like domain, (xviii) a collectin coiled-coil neck domain, (xix) an oligomerization domain, (xx) a signal domain, (xxi) an anchor domain, (xxii) a collagen-like domain, (xxiii) a fibrinogen-like domain, (xxiv) an immunoglobulin domain, (xxv) an immunoglobulin-like domain, and (xxvi) a sequence variant having at least 85% sequence identity to any one of (xi)-(xxv).

In certain aspects of this embodiment, the at least one additional domain is an immunoglobulin domain. For example, the immunoglobulin domain may comprise the amino acid sequence of SEQ ID NO:9 or a sequence variant thereof having at least 85% sequence identity to SEQ ID NO:9.

In certain aspects of this embodiment, the TLR-based engineered MTMs comprise a TLR microbe-binding domain comprising the N-terminal ligand-binding domain of any one of SEQ ID NOs:15-24 or a sequence variant thereof having at least 85% sequence identity to any one of SEQ ID NOs:15-24.

In certain aspects of this embodiment, the TLR-based engineered MTMs comprise a TLR microbe-binding domain comprising the N-terminal ligand-binding domain of any one of SEQ ID NOs:15-24 or a sequence variant thereof having at least 85% sequence identity to any one of SEQ ID NOs:15-24 and an immunoglobulin domain comprising the amino acid sequence of SEQ ID NO:9 or a sequence variant thereof having at least 85% sequence identity to SEQ ID NO:9.

### Detection Devices

As indicated above, the MTMs of the invention are used in detection devices (and related methods) to detect and/or identify microbes and microbial components in a sample. Thus, the invention includes detection devices comprising one or more MTMs selected from (i) collectin-based MTMs, (ii) ficolin-based MTMs, and (iii) toll-like receptor-based MTMs, and combinations thereof, as defined herein.

The detection device may be used for detecting a microbe or a microbial component in a sample. The detection devices of the invention or at least a component thereof are coated with MTMs or otherwise display MTMs on at least one surface of the device or component thereof such that the MTMs are exposed to a sample under conditions permitting binding of microbes or microbial components (the analyte) in the sample by the MTMs. The analyte-bound MTMs are then detected and/or measured.

The detection devices of the invention include, but are not limited to, the following: dipsticks, test strips, and any other suitable sample collection means known in the art. The dipsticks, test strips and other collection means may be comprised of one or more of filters, absorbent pads or absorbent paper, each of which may comprise natural fibers (e.g. cellulose fibers; cotton fibers) and/or synthetic fibers (e.g. polyester fibers; nylon fibers; glass-fiber material); screens; mesh; tubes; hollow fibers; membranes; and nitrocellulose. The dipsticks, test strips, and any other suitable sample collection means may be encompassed within a housing, such as a plastic housing, that protects the components, provides one or more of a sample placement window and a detection window, and provides a suitable form of the device to be held in a human hand.

In an exemplary embodiment, the dipstick, test strip or other collection means is organized in the form an LFA or a VFA. Detection devices using a LFA or VFA generally comprise a physical platform that comprises a series of rectangular strip components (though other shapes can be used), with overlapping ends, optionally applied to a backing material. The specific components include a Sample Pad 3, a Conjugate Pad 2, a Membrane 4 and an Absorbent Pad 5, one or more of which is optionally applied to a Backing Card 1. A sample is applied to the Sample Pad 3, and the force of capillary action carries the sample into the Conjugate Pad 2 where analyte in the sample can be bound by labeled detecting agents. The resulting conjugates then travel to the Membrane 4 where they are detected and/or measured.

In one embodiment, the LFA or VFA utilizes an MTM, such as an engineered form of mannose-binding ligand (MBL) linked to the Fc domain of human IgG (FcMBL), as the detecting agent (e.g. SEQ ID NO:6, 7 or 8). Use of MTMs, such as FcMBL, as the detecting agent in the LFA or VFA of the present invention allows for the detection of various pathogens (including bacteria, viruses, fungi and protozoa) in a sample, such as a blood sample from a human. When used in conjunction with Europium particles as the detectable label conjugated to the MTM detecting agent, the sensitivity of the assay is such that the assay provides both qualitative and quantitative data.

The detection devices of the invention may be used in a wide variety of applications including, but not limited to, methods of detecting the presence of a microbe or microbial component in a sample, such as the bodily fluid of a subject. Such methods include contacting a sample with a detection device of the invention under conditions that permit binding of microbes or microbial components by MTMs displayed by the detection device, thus detecting microbes or microbial components in the sample. In one aspect, the microbe is a bacteria. In another aspect, the microbe is a virus. In further aspect, the microbe is a fungus. Optionally, such methods can include one or more of the following additional steps: (i) quantifying the amount of microbe or microbial component in the sample; (ii) identifying the microbe in the sample. Suitable means for identifying the microbe are discussed below.

### BRIEF DESCRIPTION OF DRAWINGS

FIG. 1. Diagram showing the structure of exemplary collectins and ficolins.

FIG. 2. Schematics of lateral flow assay platforms of the invention. (A) and (B) indicate one example of the size and spacing of the components of the platform. (C) indicates size and spacing of the components in another example of the platform. (1) indicates the Backing Card; (2) indicates the Conjugate Pad (colored pink); (3) indicates the Sample Pad (colored light grey), (4) indicates the Membrane (colored dark grey); and (5) indicates the Absorbent Pad (colored yellow). The narrow blue strip indicates the test line. The narrow red strip indicates the control line. Approximate sizes of the different components are indicated in millimeters.

FIG. 3. Schematic of a further example of the lateral flow assay platform of the invention. (1) indicates the Backing Card; (2) indicates the Conjugate Pad; (3) indicates the Sample Pad, (4) indicates the Membrane; and (5) indicates the Absorbent Pad.

FIG. 4. Results from experiments assaying for the presence of mannan in EDTA and heparinized blood.

FIG. 5. Results from experiments assaying for the presence of LPS in heparinized blood.

FIG. 6. Results from experiments assaying for the presence of LTA in heparinized blood.

FIG. 7. Image of a detection device of the invention. The oval opening is the sample placement window or well, and the rectangular opening is the detection window.

### DETAILED DESCRIPTION OF THE INVENTION

As used herein, “a” or “an” may mean one or more. As used herein when used in conjunction with the word “comprising,” the words “a” or “an” may mean one or more than one. As used herein “another” may mean at least a second or more. Furthermore, unless otherwise required by context, singular terms include pluralities and plural terms include the singular.

As used herein, “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

### II. The Present Invention

As summarized above, the present invention is generally directed to detection devices and methods of using such devices, where the detection devices comprise microbe-targeting molecules (MTMs) and/or engineered MTMs that have the shared characteristic of binding to one or more microbe-associated molecular patterns (MAMPs). The detection devices of the invention can be used in the detection and/or identification of microbes and microbial components in a sample.

The important basis of the MTMs used in the detection devices of the invention, and the related methods, is that these constructs contact and bind microbes and microbial components in a sample based on the identity of the MAMP produced by the microbe, rather than the identity of microbe itself. While some MAMPs are produced by only a single species of microbe, other MAMPs are shared across species. Thus, while some MTMs of the invention bind to only MAMPs of a particular species of microbe, other MTMs of the invention can bind to MAMPs produced by all members of a particular class, order, family, genus or sub-genus of microbe.

As used herein, “MTM” and “engineered MTM” refers to any of the molecules described herein (or described in patents or patent application incorporated by reference) that can bind to a microbe or microbe component. Unless the context indicates otherwise, the term “MTM” is used to describe all MTMs of the invention, both naturally-occurring and engineered forms of these constructs. The terms “microbe-targeting molecule” and “microbe-binding molecule” are used interchangeably herein.

### MAMPs

Before discussing the MTMs of the invention, it will be helpful to understand the molecules to which the MTMs bind. As indicated above, each of the MTMs of the invention binds to at least one microbe-associated molecular pattern (MAMPs). Some MTMs bind at least two, at least three, at least four, at least five, or more than five MAMPs.

As used herein and throughout the specification, the term “microbe-associated molecular patterns” or “MAMPs” refers to molecules, components or motifs associated with or secreted or released by microbes or groups of microbes (whole and/or lysed and/or disrupted) that are generally recognized by corresponding pattern recognition receptors (PRRs) of the MTM microbe-binding domains defined herein. In some aspects, the MAMPs encompass molecules associated with cellular components released during cell damage or lysis. Examples of MAMPs include, but are not limited to, microbial carbohydrates (e.g., lipopolysaccharide or LPS, mannose), endotoxins, microbial nucleic acids (e.g., bacterial, fungal or viral DNA or RNA; e.g., nucleic acids comprising a CpG site), microbial peptides (e.g., flagellin), peptidoglycans, lipoteichoic acids, N-formylmethionine, lipoproteins, lipids, phospholipids or their precursors (e.g., phosphochloline), and fungal glucans.

In some aspects, microbe components comprise cell wall or membrane components known as pathogen-associated molecular patterns (PAMPs) including lipopolysaccharide (LPS) endotoxin, lipoteichoic acid, and attached or released outer membrane vesicles. In some aspects, a microbe comprises a host cell membrane and a pathogen component or a PAMP.

In some aspects, microbe components comprise damage-associated molecular patterns (DAMPs), also known as danger-associated molecular patterns, danger signals, and alarmin. These biomolecules can initiate and sustain a non-infectious inflammatory response in a subject, in contrast to PAMPs which initiate and sustain an infectious pathogen-induced inflammatory response. Upon release from damaged or dying cells, DAMPS activate the innate immune system through binding to pattern recognition receptors (PRRs). DAMPS include portions of nuclear and cytosolic proteins, ECM (extracellular matrix), mitochondria, granules, ER (endoplasmic reticulum), and plasma membrane.

In some aspects, MAMPs include carbohydrate recognition domain (CRD)-binding motifs. As used herein, the term “carbohydrate recognition domain (CRD)-binding motifs” refers to molecules or motifs that are bound by a molecule or composition comprising a CRD (i.e. CRDs recognize and bind to CRD-binding motifs). As used herein, the term “carbohydrate recognition domain” or “CRD” refers to one or more regions, at least a portion of which, can bind to carbohydrates on a surface of microbes or pathogens. In some aspects, the CRD can be derived from a lectin, as described herein. In some aspects, the CRD can be derived from a mannan-binding lectin (MBL). Accordingly, in some aspects, MAMPs are molecules, components or motifs associated with microbes or groups of microbes that are recognized by lectin-based MTMs (collectin-based MTMs) described herein that have a CRD domain. In one embodiment, MAMPs are molecules, components, or motifs associated with microbes or groups of microbes that are recognized by mannan-binding lectin (MBL).

In some aspects, MAMPs are molecules, components or motifs associated with microbes or groups of microbes that are recognized by a C-reactive protein (CRP)-based MTMs (collectin-based MTMs).

For clarity MAMPs as used herein includes microbe components such as MAMPs, PAMPs and DAMPS as defined above.

When necessary, and unless otherwise detectable without pre-treatment, MAMPs can be exposed, released or generated from microbes in a sample by various sample pretreatment methods, as discussed further herein. In some aspects, the MAMPs can be exposed, released or generated by lysing or killing at least a portion of the microbes in the sample. Without limitations, any means known or available to the practitioner for lysing or killing microbe cells can be used. Exemplary methods for lysing or killing the cells include, but are not limited to, physical, mechanical, chemical, radiation, biological, and the like. Accordingly, pre-treatment for lysing and/or killing the microbe cells can include application of one or more of ultrasound waves, vortexing, centrifugation, vibration, magnetic field, radiation (e.g., light, UV, Vis, IR, X-ray, and the like), change in temperature, flash-freezing, change in ionic strength, change in pH, incubation with chemicals (e.g. antimicrobial agents), enzymatic degradation, and the like.

### Microbes

As used herein, the term “microbe”, and the plural “microbes”, generally refers to microorganism(s), including bacteria, virus, fungi, parasites, protozoan, archaea, protists, e.g., algae, and a combination thereof. The term “microbe” encompasses both live and dead microbes. The term “microbe” also includes pathogenic microbes or pathogens, e.g., bacteria causing diseases such as sepsis, plague, tuberculosis and anthrax; protozoa causing diseases such as malaria, sleeping sickness and toxoplasmosis; and fungi causing diseases such as ringworm, candidiasis or histoplasmosis.

In some aspects, the microbe is a human pathogen, in other words a microbe that causes at least one disease in a human.

In some aspects, the microbe is a Gram-positive bacterial species, a Gram-negative bacterial species, a mycobacterium, a fungus, a parasite, protozoa, or a virus. In some aspects, the Gram-positive bacterial species comprises bacteria from the class Bacilli. In some aspects, the Gram-negative bacterial species comprises bacteria from the class Gammaproteobacteria. In some aspects, the mycobacterium comprises bacteria from the class Actinobacteria. In some aspects, the fungus comprises fungus from the class Saccharomycetes.

In some aspects, the microbe is Staphylococcus aureus, Streptococcus pyogenes, Klebsiella pneumoniae, Pseudomonas aeruginosa, Mycobacterium tuberculosis, Candida albicans, or Escherichia coli. In some aspects, the microbe is S. aureus strain 3518, S. pyogenes strain 011014, K. pneumoniae strain 631, E. coli strain 41949, P. aeruginosa strain 41504, C. albicans strain 1311, or M. tuberculosis strain H37Rv.

In some aspects, the microbe is Bartonella henselae, Borrelia burgdorferi, Campylobacter jejuni, Campylobacterfetus, Chlamydia trachomatis, Chlamydia pneumoniae, Chylamydia psittaci, Simkania negevensis, Escherichia coli (e.g., 0157:H7 and K88), Ehrlichia chafeensis, Clostridium botulinum, Clostridium perfringens, Clostridium tetani, Enterococcus faecalis, Haemophilius influenzae, Haemophilius ducreyi, Coccidioides immitis, Bordetella pertussis, Coxiella burnetii, Ureaplasma urealyticum, Mycoplasma genitalium, Trichomatis vaginalis, Helicobacter pylori, Helicobacter hepaticus, Legionella pneumophila, Mycobacterium tuberculosis, Mycobacterium bovis, Mycobacterium africanum, Mycobacterium leprae, Mycobacterium asiaticum, Mycobacterium avium, Mycobacterium celatum, Mycobacterium celonae, Mycobacterium fortuitum, Mycobacterium genavense, Mycobacterium haemophilum, Mycobacterium intracellulare, Mycobacterium kansasii, Mycobacterium malmoense, Mycobacterium marinum, Mycobacterium scrofulaceum, Mycobacterium simiae, Mycobacterium szulgai, Mycobacterium ulcerans, Mycobacterium xenopi, Corynebacterium diptheriae, Rhodococcus equi, Rickettsia aeschlimannii, Rickettsia africae, Rickettsia conorii, Arcanobacterium haemolyticum, Bacillus anthracia, Bacillus cereus, Lysteria monocytogenes, Yersinia pestis, Yersinia enterocolitica, Shigella dysenteriae, Neisseria meningitides, Neisseria gonorrhoeae, Streptococcus bovis, Streptococcus hemolyticus, Streptococcus mutans, Streptococcus pyogenes, Streptococcus pneumoniae, Staphylococcus aureus, Staphylococcus epidermidis, Staphylococcus pneumoniae, Staphylococcus saprophyticus, Vibrio cholerae, Vibrio parahaemolyticus, Salmonella typhi, Salmonella paratyphi, Salmonella enteritidis, Treponema pallidum, Human rhinovirus, Human coronavirus such as SARS-CoV-2, Dengue virus, Filoviruses (e.g., Marburg and Ebola viruses), Hantavirus, Rift Valley virus, Hepatitis B, C, and E, Human Immunodeficiency Virus (e.g., HIV-1, HIV-2), HHV-8, Human papillomavirus, Herpes virus (e.g., HV-I and HV-II), Human T-cell lymphotrophic viruses (e.g., HTLV-I and HTLV-II), Bovine leukemia virus, Influenza virus, Guanarito virus, Lassa virus, Measles virus, Rubella virus, Mumps virus, Chickenpox (Varicella virus), Monkey pox, Epstein Bahr virus, Norwalk (and Norwalk-like) viruses, Rotavirus, Parvovirus B19, Hantaan virus, Sin Nombre virus, Venezuelan equine encephalitis, Sabia virus, West Nile virus, Yellow Fever virus, causative agents of transmissible spongiform encephalopathies, Creutzfeldt-Jakob disease agent, variant Creutzfeldt-Jakob disease agent, Candida, Cryptcooccus, Cryptosporidium, Giardia lamblia, Microsporidia, Plasmodium vivax, Pneumocystis carinii, Toxoplasma gondii, Trichophyton mentagrophytes, Enterocytozoon bieneusi, Cyclospora cayetanensis, Encephalitozoon hellem, or Encephalitozoon cuniculi, among other viruses, bacteria, archaea, protozoa, and fungi. In yet other aspects, the microbe is a bioterror agent (e.g., B. anthracis, and smallpox).

As used herein, “microbe component” and “microbial component” refer to any part of a microbe such as cell wall components, cell membrane components, cell envelope components, cytosolic components, intracellular components, nucleic acid (DNA or RNA), or organelles in the case of eukaryotic microbes. In some aspects, the microbial component comprises a component from a Gram-positive bacterial species, a Gram-negative bacterial species, a mycobacterium, a fungus, a parasite, a virus, or any microbe described herein or known in the art.

### Samples

The MTMs defined herein can be used to detect the MAMPs of a microbe or a microbial component in a sample, typically a liquid sample.

A sample can include but is not limited to, a biological sample, such as a patient sample, or an animal or animal model sample; an agricultural sample; a food or beverage sample; an environmental sample; a pharmaceutical sample, such as a drug sample; an environmental sample. A biological sample can include but is not limited to, cells, tissue, peripheral blood, and a bodily fluid. Exemplary biological samples include, but are not limited to, a biopsy, a tumor sample, biofluid sample; whole blood; serum; plasma; urine; sperm; mucus; tissue biopsy; organ biopsy; synovial fluid; bile fluid; cerebrospinal fluid; mucosal secretion; nasal lavage; effusion; sweat; saliva; and/or tissue sample etc. The biological sample can be collected from any source, including, e.g., human or animal suspected of being infected or contaminated by a microbe(s).

Environmental samples include, but are not limited to, air samples, liquid samples, and dry samples. Suitable air samples include, but are not limited to, an aerosol, an atmospheric sample, and a ventilator discharge. Suitable dry samples include, but are not limited to, soil.

Pharmaceutical samples include, but are not limited to, drug material samples and therapeutic fluid samples, for example, for quality control or detection of endotoxins. Suitable therapeutic fluids include, but are not limited to, a dialysis fluid.

The source of the biological samples is not limited. As non-limiting examples, samples obtained from a human, a non-human primate, bird, horse, cow, goat, sheep, a companion animal, such as a dog, cat or rodent, or other mammal may be used.

In each of the embodiments of the invention, the analyte to be detected will vary widely based on the source of the sample and the binding agent. Non-limiting examples include pathogens, such as bacteria, viruses, fungi and protozoa.

### MTMs

As summarized above, the invention is directed to detection devices, and methods of using the devices, where the detection devices comprise “microbe-targeting molecules” (MTMs) and engineered MTMs that have the shared characteristic of binding to one or more MAMPs. The detection devices of the invention can be used in the detection and/or identification of microbes and microbe components in a sample.

MTMs distinguish and bind microbes and microbial components from a sample based on the identity of the MAMPs produced by the microbe, rather than the identity of microbe itself. While some MAMPs are produced by only a single species of microbe, other MAMPs are shared across species. Thus, while some MTMs of the invention bind to only MAMPs of a particular species of microbe, other MTMs of the invention can bind to MAMPs produced by all members of a particular class, order, family, genus or sub-genus of microbe.

As will be apparent, while the “MTMs” of the invention include naturally-occurring molecules and proteins, the “engineered MTMs” of the invention are those have been manipulated in some manner by the hand of man. As used herein and throughout the specification, the term “engineered MTM” includes any non-naturally-occurring MTM. Engineered MTMs of the invention retain the binding specificity to a MAMPs of the wild-type (i.e. naturally-occurring) molecule on which the engineered MTM is based.

The MTMs of the invention are defined based on their binding activity, therefore both naturally-occurring and engineered MTMs will comprise at least one microbe-binding domain, i.e. a domain that recognizes and binds to one or more MAMPs (including, at least two, at least three, at least four, at least five, or more) as described herein. A microbe-binding domain can be a naturally-occurring or a synthetic molecule. In some aspects, a microbe-binding domain can be a recombinant molecule.

Acceptable microbe-binding domains for use in the MTMs of the invention are limited only in their ability to recognize and bind at least one MAMP. In some aspects, the microbe-binding domain may comprise some or all of a peptide; polypeptide; protein; peptidomimetic; antibody; antibody fragment; antigen-binding fragment of an antibody; carbohydrate-binding protein; lectin; glycoprotein; glycoprotein-binding molecule; amino acid; carbohydrate (including mono-; di-; tri- and poly-saccharides); lipid; steroid; hormone; lipid-binding molecule; cofactor; nucleoside; nucleotide; nucleic acid; DNA; RNA; analogues and derivatives of nucleic acids; peptidoglycan; lipopolysaccharide; small molecule; endotoxin; bacterial lipopolysaccharide; and any combination thereof.

In particular aspects, the microbe-binding domain can be a microbe-binding domain of a lectin. An exemplary lectin is mannan binding lectin (MBL) or other mannan binding molecules. Non-limiting examples of acceptable microbe-binding domains also include microbe-binding domains from toll-like receptors, nucleotide oligomerization domain-containing (NOD) proteins, complement receptors, collectins, ficolins, pentraxins such as serum amyloid and C-reactive protein, lipid transferases, peptidoglycan recognition proteins (PGRs), and any combinations thereof In some aspects, microbe-binding domains can be microbe-binding molecules described in the International Patent Application No. WO 2013/012924, the contents of which are incorporated by reference in their entirety.

The MTMs of the invention will typically have one or more domains in addition to a microbe-binding domain. Such domains include, but are not limited to, an oligomerization domain, a signal domain, an anchor domain, a collagen-like domain, a fibrinogen-like domain, an immunoglobulin domain, and an immunoglobulin-like domain.

Engineered MTMs of the invention include, but are not limited to, MTMs identical to a naturally-occurring MTM but having at least one amino acid change in comparison to the wild-type molecule on which they are based. Such “sequence-variant engineered MTMs” have at least 51, 52, 53, 54, 55, 56, 57, 58, 59, 60, 60, 61, 62, 63, 64, 65, 66, 67, 68, 69, 70, 71, 72, 73, 74, 76, 77, 78, 79, 80, 81, 82, 83, 84, 85, 86, 87, 88, 89, 90, 91, 92, 93, 94, 95, 96, 97, 98 or 99 sequence identity, though in all cases less than 100% sequence identity, to the wild-type molecule on which they are based. The changes may be any combination of additions, insertions, deletions and substitutions, where the altered amino acids may be naturally-occurring or non-naturally-occurring amino acids, and conservative or non-conservative changes.

Engineered MTMs of the invention also include, but are not limited to, MTMs that comprise domains from two or more different MTMs, i.e. fusion proteins. Such “domain-variant engineered MTMs” have domains from 2, 3, 4, 5 or more different proteins. For example, MTMs can be a fusion protein comprising a microbe-binding domain and an oligomerization domain, or a fusion protein comprising a microbe-binding domain and a signal domain, or a fusion protein comprising a microbe-binding domain, an oligomerization domain, and signal domain, to name a few examples. In each case, the domains within a domain-variant engineered MTM are from at least two different proteins. Other examples of such MTMs include fusion proteins comprising at least the microbe-binding domain of a lectin and at least a part of a second protein or peptide, e.g., but not limited, to an Fc portion of an immunoglobulin.

Engineered MTMs of the invention further include, but are not limited to, MTMs that comprise domains from two or more different MTMs, wherein at least one of the domains is a sequence variant of the wild-type domain upon which it is based, i.e. having at least one amino acid change in comparison to the wild-type molecule on which it is based. These “sequence- and domain-variant engineered MTMs” have domains from 2, 3, 4, 5 or more different proteins, and at least one of the domains has at least 50, 51, 52, 53, 54, 55, 56, 57, 58, 59, 60, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99 sequence identity, though in all cases less than 100% sequence identity, to the wild-type domain on which it is based. The changes may be any combination of additions, insertions, deletions and substitutions, where the altered amino acids may be naturally-occurring or non-naturally-occurring amino acids, and conservative or non-conservative changes.

As non-limiting examples of the MTMs used in the detection devices of the invention, three broad categories of suitable MTMs are defined in the following paragraphs, namely: (i) collectin-based MTMs, (ii) ficolin-based MTMs, and (iii) toll-like receptor-based MTMs. It should be understood that these three categories are not the only categories of MTMs encompassed by the invention.

### Collectin-Based MTM

The MTMs used in the detection devices of the invention include collectin-based MTMs. These MTMs comprise at least one microbe-binding domain of a collectin, such as the lectin carbohydrate-recognition domain (CRD).

Collectins (collagen-containing C-type lectins) are a family of collagenous calcium-dependent lectins that function in defense, thus playing an important role in the innate immune system. They are soluble molecules comprising pattern recognition receptors (PRRs) within the microbe-binding domain that recognize and bind to particular oligosaccharide structures or lipids displayed on the surface of microbes, i.e. MAMPs of oligosaccharide origin. Upon binding of collectins to a microbe, clearance of the microbe is achieved via aggregation, complement activation, opsonization, and activation of phagocytosis.

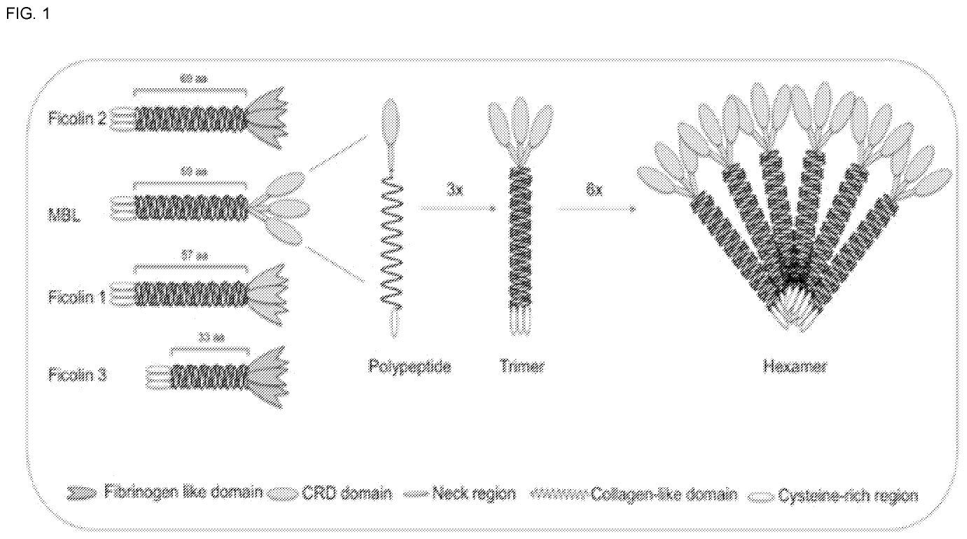

Members of the family have a common structure, characterized by four parts or domains arranged in the following N- to C-terminal arrangement: (i) a cysteine-rich domain, (ii) a collagen-like domain, (iii) a coiled-coil neck domain, and (iv) a microbe-binding domain which includes a C-type lectin domain, also termed the carbohydrate recognition domain (CRD). The functional form of the molecule is a trimer made up of three identical chains. MAMP recognition is mediated by the CRD in presence of calcium. See FIG. 1.

There are currently nine recognized members of the family: (i) mannose-binding lectin (MBL; mannan-binding lectin; e.g. SEQ ID NO:1), (ii) surfactant protein A (SP-A), (iii) surfactant protein D (SP-D), (iv) collectin liver 1 (CL-L1), (v) collectin placenta 1 (CL-P1), (vi) conglutinin collectin of 43 kDa (CL-43), (vii) collectin of 46 kDa (CL-46), (viii) collectin kidney 1 (CL-K1), and (ix) conglutinin. Each of these proteins is an MTM of the invention.

The MTMs used in the detection devices of the invention also include other collectin-based molecules that bind to one or more MAMPs, e.g. those MTMs comprising at least a portion (e.g. domain) of a lectin-based molecule in the case of an engineered MTM. As used herein, the term “collectin-based molecule” refers to a molecule comprising a microbe-binding domain derived from a collectin, such as a lectin. The term “lectin” as used herein refers to any molecule including proteins, natural or genetically modified (e.g., recombinant), that interacts specifically with saccharides (e.g., carbohydrates). The term “lectin” as used herein can also refer to lectins derived from any species, including, but not limited to, plants, animals (e.g. mammals, such as human), insects and microorganisms, having a desired carbohydrate binding specificity. Examples of plant lectins include, but are not limited to, the Leguminosae lectin family, such as ConA, soybean agglutinin, peanut lectin, lentil lectin, and Galanthus nivalis agglutinin (GNA) from the Galanthus (snowdrop) plant. Other examples of plant lectins are the Gramineae and Solanaceae families of lectins. Examples of animal lectins include, but are not limited to, any known lectin of the major groups S-type lectins, C-type lectins, P-type lectins, and I-type lectins, and galectins. In some aspects, the carbohydrate recognition domain can be derived from a C-type lectin, or a fragment thereof. C-type lectin can include any carbohydrate-binding protein that requires calcium for binding (e.g., MBL). In some aspects, the C-type lectin can include, but are not limited to, collectin, DC-SIGN, and fragments thereof. Without wishing to be bound by theory, DC-SIGN can generally bind various microbes by recognizing high-mannose-containing glycoproteins on their envelopes and/or function as a receptor for several viruses such as HIV and Hepatitis C.

Collectin-based engineered MTMs used in the detection devices of the invention are MTMs that comprise at least a microbe-binding domain of a collectin. These MTMs may also include one or more of the other domains of a collectin, e.g. a cysteine-rich domain, a collagen-like domain, and/or a coiled-coil neck domain, as well as one or more domains not typically found in a collectin, such as an oligomerization domain, a signal domain, an anchor domain, a collagen-like domain, a fibrinogen-like domain, an immunoglobulin domain, and/or an immunoglobulin-like domain. When a collectin-based engineered MTM has each of the domains of a wild-type collectin, the MTM will be a sequence-variant engineered MTM as defined above. When a collectin-based engineered MTM has fewer that all of the domains of a wild-type collectin, the MTM will be a domain-variant engineered MTM or a sequence- and domain-variant engineered MTM as defined above.

Collectin-based engineered MTMs comprise a microbe-binding domain derived from at least one carbohydrate-binding protein selected from the group consisting of: MBL; SP-A; SP-D; CL-L1, CL-P1; CL-34; CL-46; CL-K1, conglutinin; maltose-binding protein; arabinose-binding protein; glucose-binding protein; Galanthus nivalis agglutinin; peanut lectin; lentil lectin; DC-SIGN; and C-reactive protein; and any combinations thereof.

In some aspects, the MTMs and engineered MTMs used in the detection devices of the invention comprise the microbe-binding domain of a mannose-binding lectin (MBL). In some aspects, the microbe-binding domain comprises a human mannose-binding lectin (MBL; SEQ ID NO: 1). In some aspects, the microbe-binding domain comprises a MBL of a primate, mouse, rat, hamster, rabbit, or any other species as described herein. In some aspects, the microbe-binding domain comprises a portion of a human MBL (see e.g., SEQ ID NOs: 2-3). In some aspects, the microbe-binding domain comprises a plant MBL. In some aspects, the microbe-binding domain comprises a carbohydrate recognition domain (CRD) of MBL (see e.g., SEQ ID NO: 4).

MBL full length (SEQ ID NO: 1):
MSLFPSLPLL LLSMVAASYS ETVTCEDAQK TCPAVIACSS
PGINGFPGKD GRDGTKGEKG EPGQGLRGLQ GPPGKLGPPG
NPGPSGSPGP KGQKGDPGKS PDGDSSLAAS ERKALQTEMA
RIKKWLTFSL GKQVGNKFFL TNGEIMTFEK VKALCVKFQA
SVATPRNAAE NGAIQNLIKE EAFLGITDEK TEGQFVDLTG
NRLTYTNWNE GEPNNAGSDE DCVLLLKNGQ WNDVPCSTSH
LAVCEFPI
MBL without the signal sequence
(SEQ ID NO: 2):
ETVTCEDAQK TCPAVIACSS PGINGFPGKD GRDGTKGEKG
EPGQGLRGLQ GPPGKLGPPG NPGPSGSPGP KGQKGDPGKS
PDGDSSLAAS ERKALQTEMA RIKKWLTFSL GKQVGNKFFL
TNGEIMTFEK VKALCVKFQA SVATPRNAAE NGAIQNLIKE
EAFLGITDEK TEGQFVDLTG NRLTYTNWNE GEPNNAGSDE
DCVLLLKNGQ WNDVPCSTSH LAVCEFPI
Truncated MBL (SEQ ID NO: 3):
AASERKALQT EMARIKKWLT FSLGKQVGNK FFLINGEIMT
FEKVKALCVK FQASVATPRN AAENGAIQNL IKEEAFLGIT
DEKTEGQFVD LTGNRLTYTN WNEGEPNNAG SDEDCVLLLK
NGQWNDVPCS TSHLAVCEFP I
Carbohydrate recognition domain (CRD) of
MBL (SEQ ID NO: 4):
VGNKFFLTNG EIMTFEKVKA LCVKFQASVA TPRNAAENGA
IQNLIKEEAF LGITDEKTEG QFVDLIGNRL TYTNWNEGEP
NNAGSDEDCV LLLKNGQWND VPCSTSHLAV CEFPI

Alternatively or in addition, the MTMs and engineered MTMs used in the detection devices of the invention comprise the coiled-coil neck domain and a microbe-binding domain of a MBL (see, e.g. SEQ ID NO:5).

Neck + Carbohydrate recognition domain of MBL
(SEQ ID NO: 5):
PDGDSSLAAS ERKALQTEMA RIKKWLTFSL GKQVGNKFFL
TNGEIMTFEK VKALCVKFQA SVATPRNAAE NGAIQNLIKE
EAFLGITDEK TEGQFVDLTG NRLTYTNWNE GEPNNAGSDE
DCVLLLKNGQ WNDVPCSTSH LAVCEFPI

Suitable collectin-based, domain-variant engineered MTMs used in the detection devices of the invention include recombinant lectins such as FcMBL. FcMBL is a fusion protein comprising a carbohydrate recognition domain (CRD) of MBL and a portion of immunoglobulin. In some aspects, the FcMBL further comprises a neck region of MBL. In some aspects, the N-terminus of FcMBL can comprise an oligopeptide anchor domain adapted to bind a solid substrate and orient the CRD of MBL away from the solid substrate surface. See SEQ ID NOs: 6-8 for examples of FcMBLs of the invention that may be used in the detection devices, alone or in combination with each other, or in combination with other MTMs defined herein. Various genetically engineered versions of MBL (e.g., FcMBL) are described in PCT application publications WO 2011/090954 and WO 2013/012924, as well as U.S. Pat. Nos. 9,150,631 and 9,593,160, the contents of each of which are incorporated herein by reference in their entireties. Lectins and other mannan binding molecules are also described in, for example, U.S. Pat. Nos. 9,150,631 and 9,632,085, and PCT application publications WO 2011/090954, WO 2013/012924, and WO 2013/130875, the contents of all of which are incorporated herein by reference in their entireties.

Amino acid sequences for suitable engineered MTMs used in the detection devices of the invention include, but are not limited to:

FcMBL.81 (SEQ ID NO: 6):
EPKSSDKTHT CPPCPAPELL GGPSVFLFPP KPKDTLMISR
TPEVTCVVVD VSHEDPEVKF NWYVDGVEVH NAKTKPREEQ
YNSTYRVVSV LTVLHQDWLN GKEYKCKVSN KALPAPIEKT
ISKAKGQPRE PQVYTLPPSR DELTKNQVSL TCLVKGFYPS
DIAVEWESNG QPENNYKTTP PVLDSDGSFF LYSKLTVDKS
RWQQGNVFSC SVMHEALHNH YTQKSLSLSP GAPDGDSSLA
ASERKALQTE MARIKKWLTF SLGKQVGNKF FLTNGEINIT
FEKVKALCVK FQASVATPRNA AENGAIQNLI KEEAFLGITD
EKTEGQFVDL TGNRLTYTNW NEGEPNNAGS DEDCVLLLKN
GQWNDVPCST SHLAVCEFPI
AKT-FcMBL (SEQ ID NO: 7):
AKTEPKSSDK THTCPPCPAP ELLGGPSVFL FPPKPKTTLM
ISRTPEVTCV VVDVSHEDPE VKFNWYVDGV EVHNAKTKPR
EEQYNSTYRV VSVLTVLHQD WLNGKEYKCK VSNKALPAPI
EKTISKAKGQ PREPQVYTLP PSRDELTKNQ VSLTCLVKGF
YPSDIAVEWE SNGQPENNYK TTPPVLDSDG SFFLYSKLTV
DKSRWQQGNV FSCSVMHEAL HNHYTQKSLS LSPGAPDGDS
SLAASERKAL QTEMARIKKW LTFSLGKQVG NKFFLINGEI
MTFEKVKALC VKFQASVATP RNAAENGAIQ NLIKEEAFLG
ITDEKTEGQF VDLTGNRLTY TNWNEGEPNN AGSDEDCVLL
LKNGQWNDVP CSTSHLAVCE FPI
FcMBL.111 (SEQ ID NO: 8):
EPKSSDKTHT CPPCPAPELL GGPSVFLFPP KPKDTLMISR
TPEVTCVVVD VSHEDPEVKF NWYVDGVEVH NAKTKPREEQ
YNSTYRVVSV LTVLHQDWLN GKEYKCKVSN KALPAPIEKT
ISKAKGQPRE PQVYTLPPSR DELTKNQVSL TCLVKGFYPS
DIAVEWESNG QPENNYKTTP PVLDSDGSFF LYSKLTVDKS
RWQQGNVFSC SVMHEALHNH YTQKSLSLSP GATSKQVGNK
FFLTNGEEVI TFEKVKALCV KFQASVATPR NAAENGAIQN
LIKEEAFLGI TDEKTEGQFV DLTGNRLTYT NWNEGEPNNA
GSDEDCVLLL KNGQWNDVPC STSHLAVCEF PI

In some aspects, the engineered MTMs used in the detection devices of the invention comprise an amino acid sequence selected from SEQ ID NO:1-SEQ ID NO:8, or an amino acid sequence that is at least 80, 81, 82, 83, 84, 85, 86, 87, 88, 89, 90, 91, 92, 93, 94, 95, 96, 97, 98, or 99% identical to any one of SEQ ID NO:1-SEQ ID NO:8, but less than 100% identical, and that retains the microbe-binding activity of the wild-type protein.

In some aspects where the immunoglobulin domain comprises a Fc region or a fragment thereof, the Fc region or a fragment thereof can comprise at least one mutation, e.g., to modify the performance of the engineered MTMs. For example, in some aspects, a half-life of the engineered MTMs comprising an Fc region described herein can be increased, e.g., by mutating an amino acid lysine (K) at the residue 232 of SEQ ID NO: 9 to alanine (A). Other mutations, e.g., located at the interface between the CH2 and CH3 domains shown in Hinton et al (2004) J Biol Chem. 279:6213-6216 and Vaccaro C. et al. (2005) Nat Biotechnol. 23: 1283-1288, can be also used to increase the half-life of the IgG1 and thus the engineered MTMs.

SEQ ID NO: 9:
EPKSSDKTHT CPPCPAPELL GGPSVFLFPP KPKDTLMISR
TPEVTCVVVD VSHEDPEVKF NWYVDGVEVH NAKTKPREEQ
YNSTYRVVSV LTVLHQDWLN GKEYKCKVSN KALPAPIEKT
ISKAKGQPRE PQVYTLPPSR DELTKNQVSL TCLVKGFYPS
DIAVEWESNG QPENNYKTTP PVLDSDGSFF LYSKLTVDKS
RWQQGNVFSC SVMHEALHNH YTQKSLSLSP GA

The full-length amino acid sequence of the carbohydrate recognition domain (CRD) of MBL is shown in SEQ ID NO: 4. The microbe-binding domain comprising such a CDR of an engineered MTM described herein can have an amino acid sequence of about 10 to about 300 amino acid residues, or about 50 to about 160 amino acid residues. In some aspects, the microbe-binding domain can have an amino acid sequence of at least about 5, at least about 10, at least about 15, at least about 20, at least about 30, at least about 40, at least about 50, at least about 60, at least about 70, at least about 80, at least about 90, at least about 100, at least about 150 amino acid residues or more. Accordingly, in some aspects, the carbohydrate recognition domain of the engineered MTM molecule can comprise SEQ ID NO: 4. In some aspects, the carbohydrate recognition domain of the engineered MTM molecule can comprise a fragment of SEQ ID NO: 4. Exemplary amino acid sequences of such fragments include, but are not limited to, ND; EZN (where Z is any amino acid, e.g., P); NEGEPNNAGS (SEQ ID NO: 10) or a fragment thereof comprising EPN; GSDEDCVLL or a fragment thereof comprising E, and LLLKNGQWNDVPCST (SEQ ID NO: 11) or a fragment thereof comprising ND. Modifications to such CRD fragments, e.g., by conservative substitution, are also within the scope described herein. In some aspects, the MBL or a fragment thereof used in the microbe-binding domain of the engineered MTMs described herein can be a wild-type molecule or a recombinant molecule.

The exemplary sequences provided herein for the carbohydrate recognition domain of the engineered MTMs are not to be construed as limiting. For example, while the exemplary sequences provided herein are derived from a human, amino acid sequences of the same carbohydrate recognition domain in other species such as mice, rats, porcine, bovine, feline, and canine are known in the art and within the scope described herein.

### Ficolin-Based MTMs

Ficolins are a family of lectins that activate the lectin pathway of complement activation upon binding to a pathogen. Ficolins are soluble molecules comprising pattern recognition receptors (PRRs) within a microbe-binding domain that recognize and selectively bind acetylated compounds, typically N-acetylglucosamine (GlcNAc), produced by pathogens. The lectin pathway is activated by binding of a ficolin to an acetylated compound on the pathogen surface, which activates the serine proteases MASP-1 and MASP-2, which then cleave C4 into C4a and C4b, and cleave C2 into C2a and C2b. C4b and C2b then bind together to form C3-convertase of the classical pathway, leading to the eventual lysis of the target cell via the remainder of the steps in the classical pathway.

Members of the family have a common structure, characterized by three parts or domains arranged in the following N- to C-terminal arrangement: (i) a short N-terminal domain, (ii) a collagen-like domain, and (iii) a fibrinogen-like domain that makes up the microbe-binding domain. The functional form of the molecule is a trimer made up of three identical chains. See FIG. 1.

There are currently three recognized members of the family: ficolin 1 (M-ficolin), ficolin 2 (L-ficolin), and ficolin 3 (H-ficolin). Each of these proteins may be used as an MTM in the detection devices of the invention. The amino acid sequences of the human forms of the proteins are provided in the following paragraphs, with the fibrinogen-like domain underlined:

Ficolin 1 precursor - SEQ ID NO: 12 -
NP_001994.2 - Homo sapiens
MELSGATMAR GLAVLLVLFL HIKNLPAQAA DTCPEVKVVG
LEGSDKLTIL RGCPGLPGAP GPKGEAGVIG ERGERGLPGA
PGKAGPVGPK GDRGEKGMRG EKGDAGQSQS CATGPRNCKD
LLDRGYFLSG WHTIYLPDCR PLTVLCDMDT DGGGWTVFQR
RMDGSVDFYR DWAAYKQGFG SQLGEFWLGN DNIHALTAQG
SSELRVDLVD FEGNHQFAKY KSEKVADEAE KYKLVLGAFV
GGSAGNSLTG HNNNFFSTKD QDNDVSSSNC AEKFQGAWWY
ADCHASNLNG LYLMGPHESY ANGINWSAAK GYKYSYKVSE
MKVRPA
Ficolin 2 isoform a precursor - SEQ ID NO: 13 -
NP_004099.2 - Homo sapiens
MELDRAVGVL GAATLLLSFL GMAWALQAAD TCPEVKMVGL
EGSDKLTILR GCPGLPGAPG PKGEAGTNGK RGERGPPGPP
GKAGPPGPNG APGEPQPCLT GPRTCKDLLD RGHFLSGWHT
IYLPDCRPLT VLCDMDTDGG GWTVFQRRVD GSVDFYRDWA
TYKQGFGSRL GEFWLGNDNI HALTAQGTSE LRVDLVDFED
NYQFAKYRSF KVADEAEKYN LVLGAFVEGS AGDSLTFHNN
QSFSTKDQDN DLNTGNCAVM FQGAWWYKNC HVSNLNGRYL
RGTHGSFANG INWKSGKGYN YSYKVSEMKV RPA
Ficolin 3 isoform 1 precursor - SEQ ID NO: 14 -
NP_003656.2 - Homo sapiens
MDLLWILPSL WLLLLGGPAC LKTQEHPSCP GPRELEASKV
VLLPSCPGAP GSPGEKGAPG PQGPPGPPGK MGPKGEPGDP
VNLLRCQEGP RNCRELLSQG ATLSGWYHLC LPEGRALPVF
CDMDTEGGGW LVFQRRQDGS VDFFRSWSSY RAGFGNQESE
FWLGNENLHQ LTLQGNWELR VELEDFNGNR TFAHYATERL
LGEVDHYQLA LGKFSEGTAG DSLSLHSGRP FTTYDADHDS
SNSNCAVIVH GAWWYASCYR SNLNGRYAVS EAAAHKYGID
WASGRGVGHP YRRVRMMLR

In certain aspects of this embodiment, the at least one additional domain is an immunoglobulin domain. For example, the immunoglobulin domain may comprise the amino acid sequence of SEQ ID NO:9 or a sequence variant thereof having at least 85% sequence identity to SEQ ID NO:9.

The MTMs used in the detection devices of the invention also include other ficolin-based molecules that bind to one or more MAMPs (acetylated compounds for the ficolins), e.g. those MTMs comprising at least a portion (e.g. domain) of a ficolin-based molecule in the case of an engineered MTM. As used herein, the term “ficolin-based molecule” refers to a molecule comprising a microbe-binding domain derived from a ficolin. The term “ficolin” as used herein refers to any molecule including proteins, natural or genetically modified (e.g., recombinant), that interacts specifically with acetylated compounds (e.g., GlcNAc). The term “ficolin” as used herein can also refer to ficolins derived from any species, including, but not limited to, plants, animals (e.g. mammals, such as human), insects and microorganisms, having the desired binding specificity.

Ficolin-based engineered MTMs used in the detection devices of the invention are MTMs that comprise at least a microbe-binding domain of a ficolin, e.g. the fibrinogen-like domain of a ficolin. These MTMs may also include one or more of the other domains of a ficolin, e.g. a short N-terminal domain and/or a collagen-like domain, as well as one or more domains not typically found in a ficolin, such as an oligomerization domain, a signal domain, an anchor domain, a collagen-like domain, a fibrinogen-like domain, an immunoglobulin domain, and/or an immunoglobulin-like domain. When a ficolin-based engineered MTM has each of the domains of a wild-type ficolin, the MTM will be a sequence-variant engineered MTM as defined above. When a ficolin-based engineered MTM has fewer that all of the domains of a wild-type ficolin, the MTM will be a domain-variant engineered MTM or a sequence- and domain-variant engineered MTM as defined above.

Ficolin-based engineered MTMs comprise a microbe-binding domain comprising at least one fibrinogen-like domain of a ficolin selected from the group consisting of ficolin 1, ficolin 2, and ficolin 3.

In some aspects, the MTMs and engineered MTMs used in the detection devices of the invention comprise a microbe-binding domain comprising the fibrinogen-like domain of ficolin 1 of SEQ ID NO:12, optionally with an immunoglobulin domain of SEQ ID NO:9. In other aspects, the MTMs and engineered MTMs of the invention comprise a microbe-binding domain comprising the fibrinogen-like domain of ficolin 2 of SEQ ID NO:13, optionally with an immunoglobulin domain of SEQ ID NO:9. In further aspects, the MTMs and engineered MTMs of a microbe-binding domain comprising the fibrinogen-like domain of ficolin 3 of SEQ ID NO:14, optionally with an immunoglobulin domain of SEQ ID NO:9.

In some aspects, the engineered MTMs used in the detection devices of the invention comprise an microbe-binding domain having an amino acid sequence selected from SEQ ID NO:12-SEQ ID NO:14, or an amino acid sequence that is at least 80, 81, 82, 83, 84, 85, 86, 87, 88, 89, 90, 91, 92, 93, 94, 95, 96, 97, 98, or 99% identical to any one of SEQ ID NO:12-SEQ ID NO:14, but less than 100% identical, and that retains the microbe-binding activity of the wild-type protein.

In certain aspects of this embodiment, the ficolin-based engineered MTMs comprise a ficolin microbe-binding domain comprising the fibrinogen-like domain of any one of SEQ ID NOs:12, 13 and 14 or a sequence variant thereof having at least 85% sequence identity to any one of SEQ ID NOs:12, 13 and 14 and an immunoglobulin domain comprising the amino acid sequence of SEQ ID NO:9 or a sequence variant thereof having at least 85% sequence identity to SEQ ID NO:9.

In some aspects, the microbe-binding domain comprising a fibrinogen-like domain of a ficolin from a primate, mouse, rat, hamster, rabbit, or any other species as described herein.

The exemplary sequences provided herein for the ficolins are not to be construed as limiting. For example, while the exemplary sequences provided herein are derived from a human, amino acid sequences of ficolins from other species such as mice, rats, porcine, bovine, feline, and canine are known in the art and within the scope described herein.

### Toll-Like Receptor-Based MTMs

Toll-like receptors (TLRs) comprise a family of proteins that are integral to the proper functioning of the innate immune system. The proteins are type I integral membrane proteins (i.e. single-pass, membrane-spanning receptors) that are typically found on the surface of sentinel cells, such as macrophages and dendritic cells, but can also be found on the surface of other leukocytes including natural killer cells, T cells and B cells, and non-immune cells including epithelial cell, endothelial cells, and fibroblasts. After microbes have gained entry to a subject, such as a human, through the skin or mucosa, they are recognized by TLR-expressing cells, which leads to innate immune responses and the development of antigen-specific acquired immunity. TLRs thus recognize MAMPs by microbes.

Members of the family have a common structure, characterized by three parts or domains arranged in the following N- to C-terminal arrangement: (i) an N-terminal ligand-binding domain, i.e. the microbe-binding domain, (ii) a single transmembrane helix (˜20 amino acids), and (iii) a C-terminal cytoplasmic signaling domain.

The ligand-binding domain is a glycoprotein comprising 550-800 amino acid residues (depending on the identity of the TLR), constructed of tandem copies of leucine-rich repeats (LRR), which are typically 22-29 residues in length and that contains hydrophobic residues spaced at distinctive intervals. The receptors share a common structural framework in their extracellular, ligand-binding domains. The domains each adopt a horseshoe-shaped structure formed by the leucine-rich repeat motifs.

The functional form of a TLR is a dimer, with both homodimers and heterodimers being known. In the case of heterodimers, the different TLRs in the dimer may have different ligand specificities. Upon ligand binding, TLRs dimerize their ectodomains via their lateral faces, forming “m”-shaped structures. Dimerization leads to downstream signaling.

A set of endosomal TLRs comprising TLR3, TLR7, TLR8 and TLR9 recognize nucleic acids derived from viruses as well as endogenous nucleic acids in context of pathogenic events. Activation of these receptor leads to production of inflammatory cytokines as well as type I interferons (interferon type I) to help fight viral infection.

There are a number of recognized human members of the family, including TLR1, TLR2, TLR3, TLR4, TLR5, TLR6, TLR7, TLR8, TLR9, and TLR10. Each of these proteins may be used as an MTM in the detection devices of the invention. The amino acid sequences of the human forms of the proteins are provided in the following paragraphs, with the extracellular domain that comprises the N-terminal ligand-binding domain underlined:

TLR1 (toll-like receptor 1 precursor) - NCBI Reference
Sequence: NP_003254.2
(SEQ ID NO: 15)
MTSIFHFAII FMLILQIRIQ LSEESEFLVD RSKNGLIHVP KDLSQKTTIL NISQNYISEL
WTSDILSLSK LRILIISHNR IQYLDISVFK FNQELEYLDL SHNKLVKISC HPTVNLKHLD
LSFNAFDALP ICKEFGNMSQ LKFLGLSTTH LEKSSVLPIA HLNISKVLLV LGETYGEKED
PEGLQDFNTE SLHIVFPINK EFHFILDVSV KTVANLELSN IKCVLEDNKC SYFLSILAKL
QTNPKLSNLT LNNIETTWNS FIRILQLVWH TTVWYFSISN VKLQGQLDER DFDYSGTSLK
ALSIHQVVSD VFGFPQSYIY EIFSNMNIKN FTVSGTRMVH MLCPSKISPF LHLDFSNNLL
TDTVFENCGH LTELETLILQ MNQLKELSKI AEMTTQMKSL QQLDISQNSV SYDEKKGDCS
WTKSLLSLNM SSNILTDTIF RCLPPRIKVL DLHSNKIKSI PKQVVKLEAL QELNVAFNSL
TDLPGCGSFS SLSVLIIDHN SVSHPSADFF QSCQKMRSIK AGDNPFQCTC ELGEFVKNID
QVSSEVLEGW PDSYKCDYPE SYRGTLLKDF HMSELSCNIT LLIVTIVATM LVLAVTVTSL
CSYLDLPWYL RMVCQWTQTR RRARNIPLEE LQRNLQFHAF ISYSGHDSFW VKNELLPNLE
KEGMQICLHE RNFVPGKSIV ENIITCIEKS YKSIFVLSPN FVQSEWCHYE LYFAHHNLFH
EGSNSLILIL LEPIPQYSIP SSYHKLKSLM ARRTYLEWPK EKSKRGLFWA NLRAAINIKL
TEQAKK
TLR2 (toll-like receptor 2 precursor) - NCBI Reference
Sequence: NP_001305722.1
(SEQ ID NO: 16)
MPHTLWMVWV LGVIISLSKE ESSNQASLSC DRNGICKGSS GSLNSIPSGL TEAVKSLDLS
NNRITYISNS DLQRCVNLQA LVLTSNGINT IEEDSFSSLG SLEHLDLSYN YLSNLSSSWF
KPLSSLTFLN LLGNPYKTLG ETSLFSHLTK LQILRVGNMD TFTKIQRKDF AGLTFLEELE
IDASDLQSYE PKSLKSIQNV SHLILHMKQH ILLLEIFVDV TSSVECLELR DTDLDTFHFS
ELSTGETNSL IKKFTFRNVK ITDESLFQVM KLLNQISGLL ELEFDDCTLN GVGNFRASDN
DRVIDPGKVE TLTIRRLHIP RFYLFYDLST LYSLTERVKR ITVENSKVFL VPCLLSQHLK
SLEYLDLSEN LMVEEYLKNS ACEDAWPSLQ TLILRQNHLA SLEKTGETLL TLKNLTNIDI
SKNSFHSMPE TCQWPEKMKY LNLSSTRIHS VTGCIPKTLE ILDVSNNNLN LFSLNLPQLK
ELYISRNKLM TLPDASLLPM LLVLKISRNA ITTFSKEQLD SFHTLKTLEA GGNNFICSCE
FLSFTQEQQA LAKVLIDWPA NYLCDSPSHV RGQQVQDVRL SVSECHRTAL VSGMCCALFL
LILLTGVLCH RFHGLWYMKM MWAWLQAKRK PRKAPSRNIC YDAFVSYSER DAYWVENLMV
QELENFNPPF KLCLHKRDFI PGKWIIDNII DSIEKSHKTV FVLSENFVKS EWCKYELDFS
HFRLFDENND AAILILLEPI EKKAIPQRFC KLRKIMNTKT YLEWPMDEAQ REGFWVNLRA
AIKS
TLR3 (toll-like receptor 3 precursor) - NCBI Reference
Sequence: NP_003256.1
(SEQ ID NO: 17)
MRQTLPCIYF WGGLLPFGML CASSTTKCTV SHEVADCSHL KLTQVPDDLP TNITVLNLTH
NQLRRLPAAN FTRYSQLTSL DVGFNTISKL EPELCQKLPM LKVLNLQHNE LSQLSDKTFA
FCTNLTELHL MSNSIQKIKN NPFVKQKNLI TLDLSHNGLS STKLGTQVQL ENLQELLLSN
NKIQALKSEE LDIFANSSLK KLELSSNQIK EFSPGCFHAI GRLFGLFLNN VQLGPSLTEK
LCLELANTSI RNLSLSNSQL STTSNTTFLG LKWTNLTMLD LSYNNLNVVG NDSFAWLPQL
EYFFLEYNNI QHLFSHSLHG LFNVRYLNLK RSFTKQSISL ASLPKIDDFS FQWLKCLEHL
NMEDNDIPGI KSNMFTGLIN LKYLSLSNSF TSLRTLTNET FVSLAHSPLH ILNLTKNKIS
KIESDAFSWL GHLEVLDLGL NEIGQELTGQ EWRGLENIFE IYLSYNKYLQ LTRNSFALVP
SLQRLMLRRV ALKNVDSSPS PFQPLRNLTI LDLSNNNIAN INDDMLEGLE KLEILDLQHN
NLARLWKHAN PGGPIYFLKG LSHLHILNLE SNGFDEIPVE VFKDLFELKI IDLGLNNLNT
LPASVFNNQV SLKSLNLQKN LITSVEKKVF GPAFRNLTEL DMRFNPFDCT CESIAWFVNW
INETHTNIPE LSSHYLCNTP PHYHGFPVRL FDTSSCKDSA PFELFFMINT SILLIFIFIV
LLIHFEGWRI SFYWNVSVHR VLGFKEIDRQ TEQFEYAAYI IHAYKDKDWV WEHFSSMEKE
DQSLKFCLEE RDFEAGVFEL EAIVNSIKRS RKIIFVITHH LLKDPLCKRF KVHHAVQQAI
EQNLDSIILV FLEEIPDYKL NHALCLRRGM FKSHCILNWP VQKERIGAFR HKLQVALGSK
NSVH
TLR4 (toll-like receptor 4 isoform D) - NCBI Reference
Sequence: NP_612567.1
(SEQ ID NO: 18)
MNSASRLAGT LIPAMAFLSC VRPESWEPCV EVVPNITYQC NELNFYKIPD NLPFSTKNLD
LSFNPLRHLG SYSFFSFPEL QVLDLSRCEI QTIEDGAYQS LSHLSTLILT GNPIQSLALG
AFSGLSSLQK LVAVETNLAS LENFPIGHLK TLKELNVAHN LIQSFKLPEY FSNLINLEHL
DLSSNKIQSI YCTDLRVLHQ MPLLNLSLDL SLNPMNFIQP GAFKEIRLHK LTLRNNFDSL
NVMKTCIQGL AGLEVHRLVL GEFRNEGNLE KFDKSALEGL CNLTIEEFRL AYLDYYLDDI
IDLFNCLTNV SSFSLVSVTI ERVKDFSYNF GWQHLELVNC KFGQFPTLKL KSLKRLTFTS
NKGGNAFSEV DLPSLEFLDL SRNGLSFKGC CSQSDFGTTS LKYLDLSFNG VITMSSNFLG
LEQLEHLDFQ HSNLKQMSEF SVFLSLRNLI YLDISHTHTR VAFNGIFNGL SSLEVLKMAG
NSFQENFLPD IFTELRNLTF LDLSQCQLEQ LSPTAFNSLS SLQVLNMSHN NFFSLDTFPY
KCLNSLQVLD YSLNHIMTSK KQELQHFPSS LAFLNLTQND FACTCEHQSF LQWIKDQRQL
LVEVERMECA TPSDKQGMPV LSLNITCQMN KTIIGVSVLS VLVVSVVAVL VYKFYFHLML
LAGCIKYGRG ENIYDAFVIY SSQDEDWVRN ELVKNLEEGV PPFQLCLHYR DFIPGVAIAA
NIIHEGFHKS RKVIVVVSQH FIQSRWCIFE YEIAQTWQFL SSRAGIIFIV LQKVEKTLLR
QQVELYRLLS RNTYLEWEDS VLGRHIFWRR LRKALLDGKS WNPEGTVGTG CNWQEATSI
TLR5 (toll-like receptor 5 precursor) - NCBI Reference
Sequence: NP_003259.2
(SEQ ID NO: 19)
MGDHLDLLLG VVLMAGPVFG IPSCSFDGRI AFYRFCNLTQ VPQVLNTTER LLLSFNYIRT
VTASSFPFLE QLQLLELGSQ YTPLTIDKEA FRNLPNLRIL DLGSSKIYFL HPDAFQGLFH
LFELRLYFCG LSDAVLKDGY FRNLKALTRL DLSKNQIRSL YLHPSFGKLN SLKSIDFSSN
QIFLVCEHEL EPLQGKTLSF FSLAANSLYS RVSVDWGKCM NPFRNMVLEI LDVSGNGWTV
DITGNFSNAI SKSQAFSLIL AHHIMGAGFG FHNIKDPDQN TFAGLARSSV RHLDLSHGFV
FSLNSRVFET LKDLKVLNLA YNKINKIADE AFYGLDNLQV LNLSYNLLGE LYSSNFYGLP
KVAYIDLQKN HIAIIQDQTF KFLEKLQTLD LRDNALTTIH FIPSIPDIFL SGNKLVTLPK
INLTANLIHL SENRLENLDI LYFLLRVPHL QILILNQNRF SSCSGDQTPS ENPSLEQLFL
GENMLQLAWE TELCWDVFEG LSHLQVLYLN HNYLNSLPPG VFSHLTALRG LSLNSNRLTV
LSHNDLPANL EILDISRNQL LAPNPDVFVS LSVLDITHNK FICECELSTF INWLNHTNVT
IAGPPADIYC VYPDSFSGVS LFSLSTEGCD EEEVLKSLKF SLFIVCTVTL TLFLMTILTV
TKFRGFCFIC YKTAQRLVFK DHPQGTEPDM YKYDAYLCFS SKDFTWVQNA LLKHLDTQYS
DQNRFNLCFE ERDFVPGENR IANIQDAIWN SRKIVCLVSR HFLRDGWCLE AFSYAQGRCL
SDLNSALIMV VVGSLSQYQL MKHQSIRGFV QKQQYLRWPE DLQDVGWFLH KLSQQILKKE
KEKKKDNNIP LQTVATIS
TLR6 (toll-like receptor 6 precursor) - NCBI Reference
Sequence: NP_006059.2
(SEQ ID NO: 20)
MTKDKEPIVK SFHFVCLMII IVGTRIQFSD GNEFAVDKSK RGLIHVPKDL PLKTKVLDMS
QNYIAELQVS DMSFLSELTV LRLSHNRIQL LDLSVFKFNQ DLEYLDLSHN QLQKISCHPI
VSFRHLDLSF NDFKALPICK EFGNLSQLNF LGLSAMKLQK LDLLPIAHLH LSYILLDLRN
YYIKENETES LQILNAKTLH LVFHPTSLFA IQVNISVNTL GCLQLTNIKL NDDNCQVFIK
FLSELTRGST LLNFTLNHIE TTWKCLVRVF QFLWPKPVEY LNIYNLTIIE SIREEDFTYS
KTTLKALTIE HITNQVFLFS QTALYTVFSE MNIMMLTISD TPFIHMLCPH APSTFKFLNF
TQNVFTDSIF EKCSTLVKLE TLILQKNGLK DLFKVGLMTK DMPSLEILDV SWNSLESGRH
KENCTWVESI VVLNLSSNML TDSVFRCLPP RIKVLDLHSN KIKSVPKQVV KLEALQELNV
AFNSLTDLPG CGSFSSLSVL IIDHNSVSHP SADFFQSCQK MRSIKAGDNP FQCTCELREF
VKNIDQVSSE VLEGWPDSYK CDYPESYRGS PLKDFHMSEL SCHITLLIVT IGATMLVLAV
TVTSLCIYLD LPWYLRMVCQ WTQTRRRARN IPLEELQRNL QFHAFISYSE HDSAWVKSEL
VPYLEKEDIQ ICLHERNFVP GKSIVENIIN CIEKSYKSIF VLSPNFVQSE WCHYELYFAH
HNLFHEGSNN LILILLEPIP QNSIPNKYHK LKALMTQRTY LQWPKEKSKR GLFWANIRAA
FNMKLTLVTE NNDVKS
TLR7 (toll-like receptor 7 precursor) - NCBI Reference
Sequence: NP_057646.1
(SEQ ID NO: 21)
MVFPMWTLKR QILILFNIIL ISKLLGARWF PKTLPCDVTL DVPKNHVIVD CTDKHLTEIP
GGIPTNTTNL TLTINHIPDI SPASFHRLDH LVEIDFRCNC VPIPLGSKNN MCIKRLQIKP
RSFSGLTYLK SLYLDGNQLL EIPQGLPPSL QLLSLEANNI FSIRKENLTE LANIEILYLG
QNCYYRNPCY VSYSIEKDAF LNLTKLKVLS LKDNNVTAVP TVLPSTLTEL YLYNNMIAKI
QEDDFNNLNQ LQILDLSGNC PRCYNAPFPC APCKNNSPLQ IPVNAFDALT ELKVLRLHSN
SLQHVPPRWF KNINKLQELD LSQNFLAKEI GDAKFLHFLP SLIQLDLSFN FELQVYRASM
NLSQAFSSLK SLKILRIRGY VFKELKSFNL SPLHNLQNLE VLDLGTNFIK IANLSMFKQF
KRLKVIDLSV NKISPSGDSS EVGFCSNART SVESYEPQVL EQLHYFRYDK YARSCRFKNK
EASFMSVNES CYKYGQTLDL SKNSIFFVKS SDFQHLSFLK CLNLSGNLIS QTLNGSEFQP
LAELRYLDFS NNRLDLLHST AFEELHKLEV LDISSNSHYF QSEGITHMLN FTKNLKVLQK
LMMNDNDISS STSRTMESES LRTLEFRGNH LDVLWREGDN RYLQLFKNLL KLEELDISKN
SLSFLPSGVF DGMPPNLKNL SLAKNGLKSF SWKKLQCLKN LETLDLSHNQ LTTVPERLSN
CSRSLKNLIL KNNQIRSLTK YFLQDAFQLR YLDLSSNKIQ MIQKTSFPEN VLNNLKMLLL
HHNRFLCTCD AVWFVWWVNH TEVTIPYLAT DVTCVGPGAH KGQSVISLDL YTCELDLTNL
ILFSLSISVS LFLMVMMTAS HLYFWDVWYI YHFCKAKIKG YQRLISPDCC YDAFIVYDTK
DPAVTEWVLA ELVAKLEDPR EKHFNLCLEE RDWLPGQPVL ENLSQSIQLS KKTVFVMTDK
YAKTENFKIA FYLSHQRLMD EKVDVIILIF LEKPFQKSKF LQLRKRLCGS SVLEWPTNPQ
AHPYFWQCLK NALATDNHVA YSQVFKETV
TLR8 (toll-like receptor 8 isoform 1) - UniProtKB/Swiss-Prot:
Q9NR97.1
(SEQ ID NO: 22)
MENMFLQSSM LTCIFLLISG SCELCAEENF SRSYPCDEKK QNDSVIAECS NRRLQEVPQT
VGKYVTELDL SDNFITHITN ESFQGLQNLT KINLNHNPNV QHQNGNPGIQ SNGLNITDGA
FLNLKNLREL LLEDNQLPQI PSGLPESLTE LSLIQNNIYN ITKEGISRLI NLKNLYLAWN
CYFNKVCEKT NIEDGVFETL TNLELLSLSF NSLSHVPPKL PSSLRKLFLS NTQIKYISEE
DFKGLINLTL LDLSGNCPRC FNAPFPCVPC DGGASINIDR FAFQNLTQLR YLNLSSTSLR
KINAAWFKNM PHLKVLDLEF NYLVGEIASG AFLTMLPRLE ILDLSFNYIK GSYPQHINIS
RNFSKLLSLR ALHLRGYVFQ ELREDDFQPL MQLPNLSTIN LGINFIKQID FKLFQNFSNL
EIIYLSENRI SPLVKDTRQS YANSSSFQRH IRKRRSTDFE FDPHSNFYHF TRPLIKPQCA
AYGKALDLSL NSIFFIGPNQ FENLPDIACL NLSANSNAQV LSGTEFSAIP HVKYLDLTNN
RLDFDNASAL TELSDLEVLD LSYNSHYFRI AGVTHHLEFI QNFTNLKVLN LSHNNIYTLT
DKYNLESKSL VELVFSGNRL DILWNDDDNR YISIFKGLKN LTRLDLSLNR LKHIPNEAFL
NLPASLTELH INDNMLKFFN WTLLQQFPRL ELLDLRGNKL LFLTDSLSDF TSSLRTLLLS
HNRISHLPSG FLSEVSSLKH LDLSSNLLKT INKSALETKT TTKLSMLELH GNPFECTCDI
GDFRRWMDEH LNVKIPRLVD VICASPGDQR GKSIVSLELT TCVSDVTAVI LFFFTFFITT
MVMLAALAHH LFYWDVWFIY NVCLAKVKGY RSLSTSQTFY DAYISYDTKD ASVTDWVINE
LRYHLEESRD KNVLLCLEER DWDPGLAIID NLMQSINQSK KTVFVLTKKY AKSWNFKTAF
YLALQRLMDE NMDVIIFILL EPVLQHSQYL RLRQRICKSS ILQWPDNPKA EGLFWQTLRN
VVLTENDSRY NNMYVDSIKQ Y
TLR9 (toll-like receptor 9 precursor) - NCBI Reference
Sequence: NP_059138.1
(SEQ ID NO: 23)
MGFCRSALHP LSLLVQAIML AMTLALGTLP AFLPCELQPH GLVNCNWLFL KSVPHFSMAA
PRGNVTSLSL SSNRIHHLHD SDFAHLPSLR HLNLKWNCPP VGLSPMHFPC HMTIEPSTFL
AVPTLEELNL SYNNIMTVPA LPKSLISLSL SHTNILMLDS ASLAGLHALR FLFMDGNCYY
KNPCRQALEV APGALLGLGN LTHLSLKYNN LTVVPRNLPS SLEYLLLSYN RIVKLAPEDL
ANLTALRVLD VGGNCRRCDH APNPCMECPR HFPQLHPDTF SHLSRLEGLV LKDSSLSWLN
ASWFRGLGNL RVLDLSENFL YKCITKTKAF QGLTQLRKLN LSFNYQKRVS FAHLSLAPSF
GSLVALKELD MHGIFFRSLD ETTLRPLARL PMLQTLRLQM NFINQAQLGI FRAFPGLRYV
DLSDNRISGA SELTATMGEA DGGEKVWLQP GDLAPAPVDT PSSEDFRPNC STLNFTLDLS
RNNLVTVQPE MFAQLSHLQC LRLSHNCISQ AVNGSQFLPL TGLQVLDLSH NKLDLYHEHS
FTELPRLEAL DLSYNSQPFG MQGVGHNFSF VAHLRTLRHL SLAHNNIHSQ VSQQLCSTSL
RALDFSGNAL GHMWAEGDLY LHFFQGLSGL IWLDLSQNRL HTLLPQTLRN LPKSLQVLRL
RDNYLAFFKW WSLHFLPKLE VLDLAGNQLK ALTNGSLPAG TRLRRLDVSC NSISFVAPGF
FSKAKELREL NLSANALKTV DHSWFGPLAS ALQILDVSAN PLHCACGAAF MDFLLEVQAA
VPGLPSRVKC GSPGQLQGLS IFAQDLRLCL DEALSWDCFA LSLLAVALGL GVPMLHHLCG
WDLWYCFHLC LAWLPWRGRQ SGRDEDALPY DAFVVFDKTQ SAVADWVYNE LRGQLEECRG
RWALRLCLEE RDWLPGKTLF ENLWASVYGS RKTLFVLAHT DRVSGLLRAS FLLAQQRLLE
DRKDVVVLVI LSPDGRRSRY VRLRQRLCRQ SVLLWPHQPS GQRSFWAQLG MALTRDNHHF
YNRNFCQGPT AE
TLR10 (toll-like receptor 10 isoform a) - NCBI Reference
Sequence: NP_001017388.1
(SEQ ID NO: 24)
MRLIRNIYIF CSIVMTAEGD APELPEEREL MINCSNMSLR KVPADLTPAT TTLDLSYNLL
FQLQSSDFHS VSKLRVLILC HNRIQQLDLK TFEFNKELRY LDLSNNRLKS VTWYLLAGLR
YLDLSFNDFD TMPICEEAGN MSHLEILGLS GAKIQKSDFQ KIAHLHLNTV FLGFRTLPHY
EEGSLPILNT TKLHIVLPMD TNFWVLLRDG IKTSKILEMT NIDGKSQFVS YEMQRNLSLE
NAKTSVLLLN KVDLLWDDLF LILQFVWHTS VEHFQIRNVT FGGKAYLDHN SFDYSNTVMR
TIKLEHVHFR VFYIQQDKIY LLLTKMDIEN LTISNAQMPH MLFPNYPTKF QYLNFANNIL
TDELFKRTIQ LPHLKTLILN GNKLETLSLV SCFANNTPLE HLDLSQNLLQ HKNDENCSWP
ETVVNMNLSY NKLSDSVFRC LPKSIQILDL NNNQIQTVPK ETIHLMALRE LNIAFNFLTD
LPGCSHFSRL SVLNIEMNFI LSPSLDFVQS CQEVKTLNAG RNPFRCTCEL KNFIQLETYS
EVMMVGWSDS YTCEYPLNLR GTRLKDVHLH ELSCNTALLI VTIVVIMLVL GLAVAFCCLH
FDLPWYLRML GQCTQTWHRV RKTTQEQLKR NVRFHAFISY SEHDSLWVKN ELIPNLEKED
GSILICLYES YFDPGKSISE NIVSFIEKSY KSIFVLSPNF VQNEWCHYEF YFAHHNLFHE
NSDHIILILL EPIPFYCIPT RYHKLKALLE KKAYLEWPKD RRKCGLFWAN LRAAINVNVL
ATREMYELQT FTELNEESRG STISLMRTDC L

In certain aspects of this embodiment, the at least one additional domain is an immunoglobulin domain. For example, the immunoglobulin domain may comprise the amino acid sequence of SEQ ID NO:9 or a sequence variant thereof having at least 85% sequence identity to SEQ ID NO:9.

The MTMs used in the detection devices of the invention also include other TLR-based molecules that bind to one or more MAMPs, e.g. those MTMs comprising at least a portion (e.g. domain) of a TLR-based molecule in the case of an engineered MTM. As used herein, the term “TLR-based molecule” refers to a molecule comprising a microbe-binding domain (i.e. an N-terminal ligand-binding domain) derived from a TLR. The term “TLR” as used herein refers to any molecule including proteins, natural or genetically modified (e.g., recombinant), that interacts specifically with an MAMP and that has a Toll IL-1 receptor (TIR) domain in their signaling domain. The term “TLR” as used herein can also refer to TLR derived from any species, including, but not limited to, plants, animals (e.g. mammals, such as human), insects and microorganisms, having the desired binding specificity.

TLR-based engineered MTMs used in the detection devices of the invention are MTMs that comprise at least a microbe-binding domain of a TLR, e.g. the N-terminal ligand-binding domain of a TLR. These MTMs may also include one or more of the other domains of a TLR, e.g. a transmembrane helix and/or a C-terminal cytoplasmic signaling domain, as well as one or more domains not typically found in a TLR, such as an oligomerization domain, a signal domain, an anchor domain, a collagen-like domain, a fibrinogen-like domain, an immunoglobulin domain, and/or an immunoglobulin-like domain. When a TLR-based engineered MTM has each of the domains of a wild-type TLR, the MTM will be a sequence-variant engineered MTM as defined above. When a TLR-based engineered MTM has fewer that all of the domains of a wild-type TLR, the MTM will be a domain-variant engineered MTM or a sequence- and domain-variant engineered MTM as defined above.

TLR-based engineered MTMs comprise a microbe-binding domain comprising at least one N-terminal ligand-binding domain of a TLR selected from the group consisting of TLR1, TLR2, TLR3, TLR4, TLR5, TLR6, TLR7, TLR8, TLR9, and TLR10.

In some aspects, the MTMs and engineered MTMs used in the detection devices of the invention comprise a microbe-binding domain comprising the N-terminal ligand-binding domain of TLR1 of SEQ ID NO:15, or the N-terminal ligand-binding domain of TLR2 of SEQ ID NO:16, or the N-terminal ligand-binding domain of TLR3 of SEQ ID NO:17, or the N-terminal ligand-binding domain of TLR4 of SEQ ID NO:18, or the N-terminal ligand-binding domain of TLR5 of SEQ ID NO:19, or the N-terminal ligand-binding domain of TLR6 of SEQ ID NO:20, or the N-terminal ligand-binding domain of TLR7 of SEQ ID NO:21, or the N-terminal ligand-binding domain of TLR8 of SEQ ID NO:22, or the N-terminal ligand-binding domain of TLR9 of SEQ ID NO:23, or the N-terminal ligand-binding domain of TLR10 of SEQ ID NO:24. In each of these examples, MTMs and engineered MTMs may further comprise an immunoglobulin domain of SEQ ID NO:9.

In some aspects, the engineered MTMs used in the detection devices of the invention comprise an microbe-binding domain having an amino acid sequence selected from SEQ ID NO:15-SEQ ID NO:24, or an amino acid sequence that is at least 80, 81, 82, 83, 84, 85, 86, 87, 88, 89, 90, 91, 92, 93, 94, 95, 96, 97, 98, or 99% identical to any one of SEQ ID NO:15-SEQ ID NO:24, but less than 100% identical, and that retains the microbe-binding activity of the wild-type protein.

In certain aspects of this embodiment, the TLR-based engineered MTMs comprise a TLR microbe-binding domain comprising the N-terminal ligand-binding domain of any one of SEQ ID NOs:15-24 or a sequence variant thereof having at least 85% sequence identity to any one of SEQ ID NOs:15-24 and an immunoglobulin domain comprising the amino acid sequence of SEQ ID NO:9 or a sequence variant thereof having at least 85% sequence identity to SEQ ID NO:9.

In some aspects, the microbe-binding domain comprising a N-terminal ligand-binding domain of a TLR from a primate, mouse, rat, hamster, rabbit, or any other subject as described herein.

The exemplary sequences provided herein for the TLRs are not to be construed as limiting. For example, while the exemplary sequences provided herein are derived from a human, amino acid sequences of TLRs from other species such as mice, rats, porcine, bovine, feline, and canine are known in the art and within the scope described herein.

In some further aspects, the MTMs used in the detection devices of the invention are those described in at least one of the following: U.S. provisional application Nos. 61/296,222, 61/508,957, 61/604,878, 61/605,052, 61/605,081, 61/788,570, 61/846,438, 61/866,843, 61/917,705, 62/201,745, 62/336,940, 62/543,614; PCT application numbers PCT/US2011/021603, PCT/US2012/047201, PCT/US2013/028409 , PCT/US2014/028683, PCT/US2014/046716, PCT/US2014/071293, PCT/US2016/045509, PCT/US2017/032928; U.S. patent application Ser. Nos. 13/574,191, 14/233,553, 14/382,043, 14/766,575, 14/831,480, 14/904,583, 15/105,298, 15/415,352, 15/483,216, 15/668,794, 15/750,788, 15/839,352, 16/059,799, 16/302,023, 16/553,635; and U.S. Pat. Nos. 9,150,631, 9,593,160, 9,632,085, 9,791,440, and 10,435,457; the contents of each of which are incorporated by reference herein in their entireties.

### Labels

The MTMs used in the detection devices of the present invention may be labeled to allow them to be detected after binding to microbes or microbial components from a sample. The identity of the detectable label is limited only in that it can be discerned by the human eye or via a detector in the context of the detection device. Suitable detectable labels include colored or fluorescent particles, such a Europium particles or colloidal gold. Other acceptable labels include latex, which may itself be tagged with colored or fluorescent dyes, and magnetic or paramagnetic components. A further detectable label is a plasmonic fluor, wherein instead of assaying for a color change, one detects fluorescence. Ultrabright fluorescent nanolabels can also be used to improve the limit of detection in the detection devices of the invention, compared with conventional fluorophores.

### Detection Devices

As suggested above, the MTMs are used in detection devices (and related methods) to detect and/or identify microbes and microbial components in a sample.

The detection devices of the invention are limited only in that (i) they are devices (or components thereof) that may be used to detect and/or identify microbes and microbial components in a sample, and (ii) they contain one or more MTMs of the invention. At least one surface of the device (or a component thereof) is coated with MTMs of the invention, or otherwise display MTMs such that the MTMs are exposed to a sample under conditions permitting binding of microbes or microbial components in the sample by the MTMs.

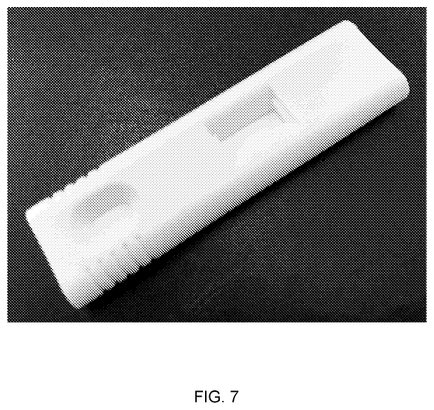

The detection devices of the invention include, but are not limited to, the following: dipsticks, test strips, and any other suitable sample collection means known in the art. The dipsticks, test strips and other collection means may be comprised of one or more of natural fibers (e.g. cellulose fibers; cotton fibers) and/or synthetic fibers (e.g. polyester fibers (e.g. Conjugate Pads 6614, Ahlstrom-Munksjö, Helsinki, Finland); nylon fibers; glass-fiber material (e.g. LyPore® 9389 pads, Lyndall Inc., Rochester, NH)); screens; mesh; tubes; hollow fibers; membranes (e.g. Fusion-5 membranes); nitrocellulose (e.g. UniSart® Nitrocellulose Membrane CN140, Satorius Inc., Gottingen, Germany); and other substrates commonly utilized in lateral flow assay formats, and any combinations thereof. The dipsticks, test strips, and any other suitable sample collection means may be encompassed within a housing, such as a plastic housing, that protects the components, provides one or more of a sample placement window and a detection window, and provides a suitable form of the device to be held in a human hand (see FIG. 7).

Exemplary diagnostic devices that comprise at least one component having a surface that displays MTMs of the invention include a lateral flow assay (LFA) and a vertical flow assay (VFA). LFAs and VFAs generally comprise a physical platform that comprises a series of rectangular strip components (though other shapes can be used), with overlapping ends, optionally applied to a backing material. The specific components include a Sample Pad 3, a Conjugate Pad 2, a Membrane 4 and an Absorbent Pad 5, one or more of which is optionally applied to a Backing Card 1. A sample is applied to the Sample Pad 3, and the force of capillary action carries the sample into the Conjugate Pad 2 where analyte in the sample can be bound by labeled detecting agents. The resulting conjugates then travel to the Membrane 4 where they are detected and/or measured.

In one embodiment, the LFA or VFA utilizes an MTM, such as an engineered form of mannose-binding ligand (MBL) linked to the Fc domain of human IgG (FcMBL), as the detecting agent (e.g. SEQ ID NO:6, 7 or 8). Use of MTMs, such as FcMBL, as the detecting agent in the LFA or VFA of the present invention allows for the detection of various pathogens (including bacteria, viruses, fungi and protozoa) in a sample, such as a blood sample from a human. When used in conjunction with Europium particles as the detectable label conjugated to the MTM detecting agent, the sensitivity of the assay is such that the assay provides both qualitative and quantitative data.

The detection devices of the invention may be used in a wide variety of screening applications including, but not limited to, methods of detecting the presence of a microbe or microbial component in a sample, such as a bodily fluid of a subject. Such methods include contacting a sample with a detection device of the invention under conditions that permit binding of microbes or microbial components by MTMs displayed by the detection device, thus detecting microbes or microbial components in a sample. In one aspect, the microbe is a bacteria. In another aspect, the microbe is a virus. In further aspect, the microbe is a fungus. In further aspect, the microbe is a protozoan. Optionally, such methods can include one or more of the following additional steps: (i) quantifying the amount of microbe or microbial component in the sample; (ii) identifying the microbe in the sample. Suitable means for identifying the microbe are discussed below.

The MTMs are coated and/or immobilized onto at least one surface of the device, or a component of the device, such that the MTMs either coat all surfaces of the device or component, or only selected portions of the device or component. The “surface” can be an exterior surface, such as when a component of the device is a flat, impermeable surface, or the “surface” can be the surface of elements that comprise the component. For example, when the component is a filter, absorbent pad or absorbent paper, the “surface” will be understood to be the surface of the individual cellulose or synthetic fibers that comprise the filter, pad or paper. Thus, the MTMs can be located within a filter, pad or paper, yet still be considered to be on a “surface” of the filter, pad or paper.

Immobilization (via coating) of MTMs onto a surface can be either non-specific (e.g., by adsorption to the surface) or specific (e.g. where another molecule, such as a linker, immobilized on the surface is used to capture the MTM). MTMs may be linked to the surface through one or more linkers which may be cleavable to accommodate release or elution of the bound target molecules for subsequent analysis. MTMs may attach to the one or more surfaces though a covalent linking process. Substrate linkage may be accomplished through, for example, biotin-avidin binding, 1-Ethyl-3-[3-dimethylaminopropyl]carbodiimide hydrochloride (EDC or EDAC), hydroxybenzotriazole (HOBT), N-Hydroxysuccinimide (NHS), 2-(1H-7-Azabenzotriazol-1-yl)-1,1,3,3-tetramethyl uronium hexafluorophosphate methanaminium (HATU), silanization, surface activation through plasma treatment, and the like.

In some embodiments, the surface is fabricated or coated with a material prior to being coated by MTMs, where the material is one or more of polydimethylsiloxane, polyimide, polyethylene terephthalate, polymethylmethacrylate, polyurethane, polyvinylchloride, polystyrene polysulfone, polycarbonate, polymethylpentene, polypropylene, a polyvinylidine fluoride, polysilicon, polytetrafluoroethylene, polysulfone, acrylonitrile butadiene styrene, polyacrylonitrile, polybutadiene, poly(butylene terephthalate), poly(ether sulfone), poly(ether ether ketones), poly(ethylene glycol), styrene-acrylonitrile resin, poly(trimethylene terephthalate), polyvinyl butyral, polyvinylidenedifluoride, poly(vinyl pyrrolidone), and any combination thereof.

In some embodiments, MTMs can be conjugated to the surface by methods well known in the art for conjugating peptides with other molecules. For example, Hermanson, BIOCONJUGATE TECHNIQUES (2nd Ed., Academic Press (2008)) and Niemeyr, Bioconjugation Protocols: Strategies & Methods, in METHODS IN MOLECULAR BIOLOGY (Humana Press, 2004), provide a number of methods and techniques for conjugating peptides to other molecules. de Graaf, et al., 20 Biocojugate Chem. 1281 (2009), provides a review of site-specific introduction of non-natural amino acids into peptides for conjugation.

Alternatively, the surface can be functionalized to include binding molecules that bind selectively with the MTMs. The binding molecule can be bound covalently or non-covalently on the surface. As used herein, the term “binding molecule” refers to any molecule that is capable of specifically binding MTMs, as defined herein. Representative examples of binding molecule include, but are not limited to, antibodies, antigens, lectins, proteins, peptides, nucleic acids (DNA, RNA, PNA and nucleic acids that are mixtures thereof or that include nucleotide derivatives or analogs); receptor molecules, such as the insulin receptor; ligands for receptors (e.g., insulin for the insulin receptor); and biological, chemical or other molecules that have affinity for another molecule, such as biotin and avidin. The binding molecules need not comprise an entire naturally occurring molecule but may consist of only a portion, fragment or subunit of a naturally or non-naturally occurring molecule, as for example the Fab fragment of an antibody. The binding molecule may further comprise a marker that can be detected.

The binding molecule can be conjugated to the surface using any of a variety of methods known to those of skill in the art. The binding molecule can be coupled or conjugated to surface of the substrate covalently or non-covalently. Covalent immobilization may be accomplished through, for example, silane coupling. See, e.g., Weetall, 15 Adv. Mol. Cell Bio. 161 (2008); Weetall, 44 Meths. Enzymol. 134 (1976). The covalent linkage between the binding molecule and the surface can also be mediated by a linker. The non-covalent linkage between the binding molecule and the surface can be based on ionic interactions, van der Waals interactions, dipole-dipole interactions, hydrogen bonds, electrostatic interactions, and/or shape recognition interactions.

As used herein, the term “linker” means a molecular moiety that connects two parts of a composition. Peptide linkers may affect folding of a given fusion protein, and may also react/bind with other proteins, and these properties can be screened for by known techniques. Example linkers, in addition to those described herein, include is a string of histidine residues, e.g., His6; sequences made up of Ala and Pro, varying the number of Ala-Pro pairs to modulate the flexibility of the linker; and sequences made up of charged amino acid residues e.g., mixing Glu and Lys. Flexibility can be controlled by the types and numbers of residues in the linker. See, e.g., Perham, 30 Biochem. 8501 (1991); Wriggers et al., 80 Biopolymers 736 (2005). Chemical linkers may comprise a direct bond or an atom such as oxygen or sulfur, a unit such as NH, C(O), C(O)NH, SO, SO₂, SO₂NH, or a chain of atoms, such as substituted or unsubstituted C₁-C₆ alkyl, substituted or unsubstituted C₂-C₆ alkenyl, substituted or unsubstituted C₂-C₆ alkynyl, substituted or unsubstituted C₆-C₁₂ aryl, substituted or unsubstituted C₅-C₁₂ heteroaryl, substituted or unsubstituted C₅-C₁₂ heterocyclyl, substituted or unsubstituted C₃-C₁₂ cycloalkyl, where one or more methylenes can be interrupted or terminated by O, S, S(O), SO₂, NH, or C(O).

### Lateral Flow Assay

In one aspect of the invention, the detection device comprises a lateral flow assay (LFA). An LFA is a paper- and/or membrane-based platform for detecting and/or quantifying analytes in complex sample mixtures. Lacking mechanical parts, a sample to be assayed is placed on one end of the platform, the analyte moves via capillary action, a signal is generated, and the results are displayed within 5-30 min. Depending on the elements of the detecting system used in the assay, results can be determined with the unaided eye (as in a home pregnancy test) or through the use of a detection device (e.g., a lateral flow reader for the detection of visible colorimetric or fluorescent signals). Because the analyte moves via capillary action, the physical orientation of the assay may be unimportant in some instances. Therefore, a vertical flow assay (VFA) made be used as an alternative to an LFA. The components of a VFA may be the same as the components comprising an LFA.

LFA can be used where rapid analysis is needed, for example, in hospitals, physician's offices and clinical laboratories, and they can be used to detect and even measure the amounts of selected chemical, compounds and proteins in a sample, such as antigens and antibodies, chemicals, toxins, pathogens and pathogen debris. A nearly unlimited source of samples can be assayed via LFAs, including biological samples such as urine, saliva, sweat, serum, plasma, and whole blood, and environmental samples. LFA-based tests can also be used in veterinary applications, quality control applications, product safety applications (e.g., food production), and environmental health and safety applications.

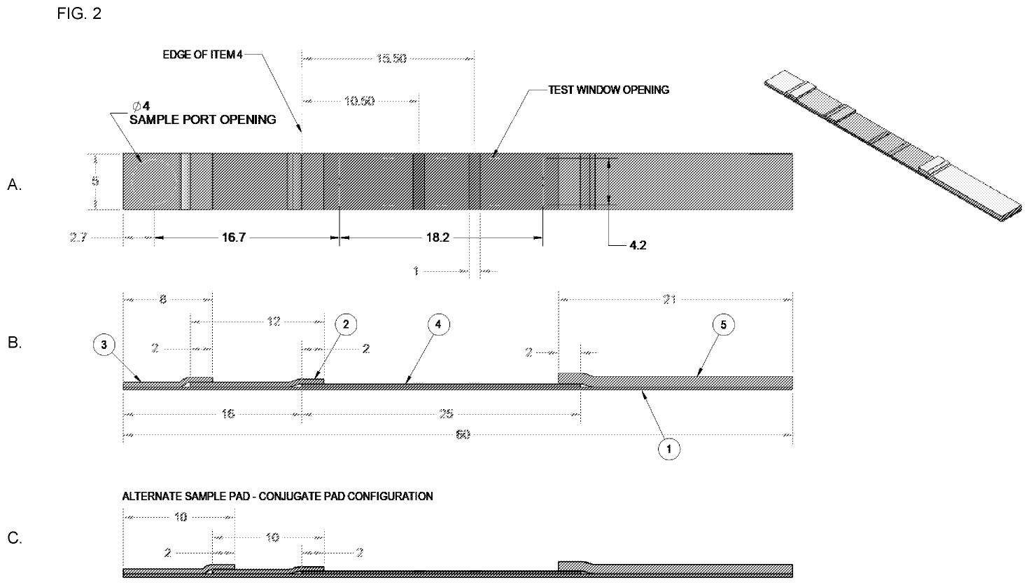

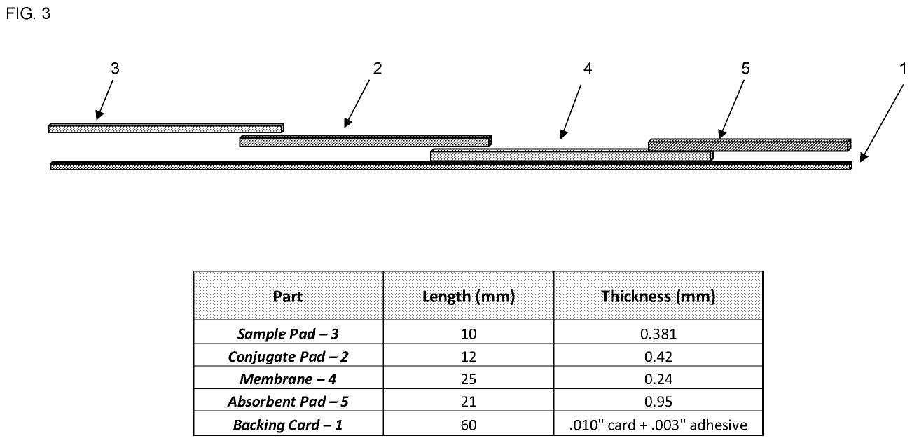

As discussed in the review by Koczula K. M. and A. Gallotta (Essays Biochem. 60(1):111-120 (2016)), incorporated by reference in its entirety herein, the operation of an LFA comprises the movement of a liquid sample containing the analyte of interest via capillary action through various zones of polymeric strips arranged on the platform. The strips contain molecules that can interact with the analyte if it is present in the sample. Lateral flow platforms of the present invention consist of overlapping strips of different materials that are optionally mounted on a Backing Card 1 (see FIGS. 2 and 3). The sample is applied to the Sample Pad 3, which may be impregnated with buffer salts and surfactants that make the sample suitable for interaction with the detection system. Alternatively, or in addition, a running buffer may be added after the sample to aid in movement of the sample through the strips of the platform.

The sample migrates from the Sample Pad 3 into and through the Conjugate Pad 2, which contains one or more MTMs (binding agents) that will recognize and bind to the target analyte. These MTM binding agents are typically conjugated to detectable labels, e.g. colored or fluorescent particles. The sample, now comprising target analyte conjugated to a labeled MTM, migrates from the Conjugate Pad 2 into a porous Membrane 4.

The Membrane 4 comprises nitrocellulose, for example, and it may include a Detection Zone. The Detection Zone includes capture components present in the shape of one or more lines, typically a Test Line and a Control Line (see FIG. 2A). The Test Line and Control Line occupy separate locations on the Membrane 4. The capture components react with the analyte bound to the labeled MTM. Recognition by the capture components of the analyte bound to the labeled MTM allows the label to remain static and thus detectable on the Test Line. A similar detectable signal on the Control Line indicates adequate sample flow through the strips comprising the platform and proper functioning of the assay.

To ensure adequate and continuous movement of the sample across the strips comprising the platform, an Absorbent Pad 5 is placed into contact with the Membrane 4 and attached to the end of the strip. The Absorbent Pad 5 also serves to wick excess reagents and to prevent backflow of the sample.

The detectable signals (i.e. the labeled MTM) present on the Test Line and the Control Line can be detected and/or measured using the unaided eye or using a detection device (e.g., a lateral flow reader for the detection of visible colorimetric or fluorescent signals).

While the general features of the LFA have now been described, the specific aspects of the LFA of the present invention are provided in the following paragraphs.

### Assay Components

The LFA of the present invention is platform comprising six main components. These components are described in the following paragraphs.

Sample Pad 3—As indicated above, the Sample Pad 3 is the portion of the platform onto which the sample is applied. The Sample Pad 3 may be impregnated with buffer salts, proteins, antibodies, surfactants, sugars (e.g., glucose, sucrose, and trehalose) and other materials to control the flow rate of the sample, sample treatment and to make target analyte of the sample suitable for interaction with the detection system. Alternatively, or in addition, a running buffer may be added to the Sample Pad 3 after the sample has been applied. A running buffer serves to move analytes present in the sample through the components of the platform via capillary action. Running buffer may also facilitate the binding of the analyte to the MTMs. A section of the Sample Pad 3 may be marked (e.g. “Sample Well”) to indicate where to apply the sample and the optional running buffer.

Depending on the material used for the Sample Pad 3, pores may be present in the material comprising the Sample Pad 3 which serve to filter out and exclude undesired materials from flowing onto the Conjugate Pad 2, for example, to remove impurities such as particles or red blood cells from blood samples.

Suitable materials that may be used as the Sample Pad 3 include, but are not limited to, pads comprising glass-fiber material such as LyPore® 9389 pads from Lyndall Inc., Rochester, NH or Ahlstrom. Other suitable materials include but are not limited to cellulose fiber, nylon fiber, and Fusion-5 membranes. Sample Pad 3 may be coated, where coatings may comprise surfactants, blockers, proteins (e.g., albumin, casein), and sugars. Suitable lengths and widths of the Sample Pad 3 depend on the overall size of the platform, but a convenient range of lengths includes about 5-15 mm, with about 10 mm being exemplary, and a convenient range of widths include about 1-10 mm, with about 5 mm being exemplary.

Conjugate Pad 2—One end of the Sample Pad 3 is applied onto and thus overlaps with one end of the Conjugate Pad 2 (see FIGS. 2 and 3). Depending on the size of these two strips, the amount of overlap will be about 20% of the length of one of the strips, but may also range from about 1% to about 50%. If the Sample Pad 3 is about 10 mm in length and the Conjugate Pad 2 is about 12 mm in length, an overlap of about 2 mm is suitable. In one embodiment of the invention, the Sample Pad 3 is about 10 mm in length, the Conjugate Pad 2 is about 12 mm in length, the width of the pads is about 5 mm, and the overlap is about 2 mm. The Sample Pad 3 may be adhered to the Conjugate Pad 2 using lamination via Backing Pad 1.

Conjugate Pad 2 is the portion of the platform that includes the one or more labeled MTMs. The labeled MTMs comprise (i) a MTM that is specific to the target analyte and (ii) a detectable label conjugated to the MTM. Optionally, one or more additives may be included in the Conjugate Pad 2, including but not limited to, sugars (e.g., glucose and sucrose), surfactants (e.g., Tween-20, NP-40), biopolymers (e.g., PVP and PVA), and proteins (e.g., albumin and casein).

Suitable MTMs include MTMs having specificity for molecules or groups of molecules, as well as MTMs that recognize broad groups or class of molecules, as defined herein.

Suitable detectable labels include colored or fluorescent particles, such a Europium particles or colloidal gold, that can be conjugated to the MTMs and detected once the analyte is on the Test Line and Control Line. Other acceptable labels include latex, which may itself be tagged with colored or fluorescent dyes, and magnetic or paramagnetic components.

Depending on the identity of the MTM and detectable label, the Conjugate Pad 2 maintains the functional stability of these molecules until the assay is performed.

Suitable materials that may be used as the Conjugate Pad 2 include, but are not limited to, pads comprising glass fibers, polyester fibers, Fusion-5 membrane and combinations thereof, such as the polyester fiber Conjugate Pads 6614 of Ahlstrom-Munksjö, Helsinki, Finland. Suitable lengths and widths of the Conjugate Pad 2 depend on the overall size of the platform, but a convenient range of lengths includes about 5-20 mm, with about 12 mm being exemplary, and a convenient range of widths include about 1-10 mm, with about 5 mm being exemplary.

In an exemplary embodiment of the invention, (i) FcMBL (SEQ ID Nos:6-8) conjugated with Europium particles (FcMBL-Eu) and (ii) biotinylated MBL are both used as binding agents in the Conjugate Pad 2. These MTM binding agents may be located at the same or separate locations on the Conjugate Pad 2. When the target analyte is present in the sample, a complex forms between the target analyte, FcMBL-Eu and biotinylated MBL, where the analyte is sandwiched between the two different forms of MTM. In another exemplary embodiment of the invention, the analyte is sandwiched between the same forms of MTM.

Membrane 4—The end of Conjugate Pad 2 not adhered to the Sample Pad 3 is applied onto and thus overlaps with one end of the Membrane 4 (see FIGS. 2 and 3). Depending on the size of these two elements, the amount of overlap will be about 20% of the length of one of the strips, but may also range from about 1% to about 50%. If the Conjugate Pad 2 is about 12 mm in length and the Membrane 4 is about 25 mm in length, an overlap of about 2 mm is suitable. In one embodiment of the invention, the Conjugate Pad 2 is about 12 mm in length, the Membrane 4 is about 25 mm in length, the width of the pads is about 5 mm, and the overlap is of about 2 mm. The Conjugate Pad 2 may be adhered to the Membrane 4 using lamination via Backing Pad 1.

The Membrane 4 is the portion of the platform that includes the Detection Zone. The Detection Zone includes capture components present in the shape of one or more lines, for example, a Test Line and a Control Line (see FIG. 2A). These capture components react with the analyte bound to the labeled MTM. Recognition by the capture components of the sample analyte bound to the labeled MTM maintains the analyte-labeled binding agent complex in a static location on the Test Line. A similar detectable signal on the Control Line indicates adequate sample flow through the strips comprising the platform and proper functioning of the assay.

The Test Line will generally be located on the Membrane 4 such that it encounters the analyte-labeled MTM complex first. The sample will continue to move across the Membrane 4 where the analyte-labeled MTM complex will then encounter the Control Line. Suitable locations and sizes of the Lines will vary and depend on the overall size of the other components of the platform, but when the Membrane 4 is about 25 mm in length, the Test Line may be located about 1.2 cm from the end of the Membrane 4 adhered to the Conjugate Pad 2, and the Control Line may be located about 1.6 cm from the end of the Membrane 4 adhered to the Conjugate Pad 2. Both the Test Line and Control Line may be about 0.8 mm in width under these circumstances.

Suitable capture components include, but are not limited to, streptavidin and polystreptavidin (where the labeled MTM is biotinylated), and any of the binding agents described herein.

Membrane 4 may comprise nitrocellulose, such as UniSart® Nitrocellulose Membrane CN140 from Satorius Inc., Gottingen, Germany, and Fusion-5 Membrane. Suitable lengths and widths of the Membrane 4 depend on the overall size of the platform, but a convenient range of lengths includes about 20-30 mm, with about 25 mm being exemplary, and a convenient range of widths include about 1-10 mm, with about 5 mm being exemplary. Membrane 4 can be selected based on binding and flow characteristics to achieve the optimum sensitivity within the detection time. In some aspects, Membrane 4 can be blocked with proteins to reduce a non-specific signal.

Absorbent Pad 5—Onto the end of Membrane 4 not adhered to Conjugate Pad 2 is applied one end of the Absorbent Pad 5 (see FIGS. 2 and 3). Depending on the size of these two elements, the amount of overlap will be about 20% of the length of one of the strips, but may also range from about 1% to about 50%. If the Membrane 4 is about 25 mm in length and the Absorbent Pad 5 about 21 mm in length, an overlap of about 2 mm is suitable. In one embodiment of the invention, the Membrane 4 is about 25 mm in length, the Absorbent Pad 5 about 21 mm in length, the width of the pads is about 5 mm, and the overlap is of about 2 mm. The Absorbent Pad 5 is adhered to the Membrane 4 using lamination via Backing Pad 1.

The Absorbent Pad 5 serves to wick the sample through the different components of the platform and to collect the processed sample. Absorbent pads may comprise cellulose filters, cotton filters or Fusion-5. Suitable cellulose filters include Whatman 243.

Backing Card 1—Each of the four components of the platform may be adhered to the Backing Card 1 using acrylic pressure sensitive adhesives or any adhesive that is non-reactive and stable during operation. In some aspects, a non-fluorescent backing card may be used for the fluorescent assay (see FIGS. 2 and 3).

Suitable materials for use as the Backing Card 1 include vinyl.

The backing card will generally be the same length and width of the other, joined components. In one embodiment of the invention, the Backing Card 1 is about 60 mm in length and a width of about 5 mm.

Housing—The combination of strips may be housed in a cassette (i.e. housing) which contains a cut-out for a sample placement window or well (over the Sample Pad 3) and a cut-out for a detection window (over the Detection Zone) where Test and Control Lines can be seen (see FIG. 7). The cassette is comprised of plastic or other suitable material. that protects the components.

While the detection devices, in the context of a LFA, are described as a hand-held devices comprising a single platform, the invention is also scalable and includes larger detection devices, either arranged in a lateral or vertical format, comprising a few or many platforms, that can be used to test multiple samples in a short period of time.

### Method of Using the LFA

The FLA platforms of the invention may be used in the following manner.

### Sample Preparation

Depending on the identity of the sample to be assayed using the LFA of the invention, an aliquot of the sample may be applied directed to the Sample Pad 3 or it may first be processed. When the sample is whole blood, plasma, or serum, the sample may first be treated with EDTA and/or the sample may be heparinized. The sample will typically be at room temperature (18 to 30° C.) before it is added to the sample. The sample size will vary depending on the size of the components in the platform, but when the exemplary platforms of the invention described herein are used, the sample size will be about 20 μl.

The sample can be applied to the Sample Pad 3 using any suitable device, although the use of a capillary tube is helpful when small volumes (e.g. 20 μl) are applied.

### Buffer Application

In some applications, especially when a small sample size is applied to the Sample Pad 3 (e.g. 20 μl), it can be helpful to apply a running buffer to the Sample Pad 3 in the same location to which the sample as applied. The use of a running buffer will help to ensure that the analyte in the same is carried through each of the strips that make up the platform, and into the Absorbent Pad 5.

Suitable running buffers will depend on the identity of the sample being analyzed, but when the sample is blood or a component of blood, the following running buffer is suitable: 12 mM HEPES, pH, 7.0, 1.2% Tween-20, 0.6 M NaCl, 10 mM CaCl₂. A volume of 75 μl is suitable when the sample volume is around 20 μl.

### Reaction Time

The reaction time, i.e. the period of time over which the sample moves across the platform, will vary depending on factors such as the source and size of the sample, whether a running buffer is used, and the size and composition of the components of the platform. However, for the exemplary platforms defined herein, a reaction time of about 10 minutes it suitable.

### Detecting and Measuring Signal

Appropriate means for detecting and/or measuring the amount of analyte present on the Test Line or Control Line will vary depending on factors such as the source and size of the sample, the amount of analyte in the sample, the identity of the analyte, the identity and amount of the MTM binding agent, the identity and amount of the detectable signal (i.e. label on the MTM), and the identity and amount of the capture components. However, suitable means include the unaided eye and mechanical means designed to detect and measure the label. When the detectable label is europium particles, an fluorescent reader capable of Eu signal measurement may be used, for example, an Axxin AX-2X-S Multi-Spectral Instrument (Axxin, Fairfield, Australia).

The detecting and/or measuring of the signal may also be accomplished using a mobile telephone application, such as a signal reader that utilizes components of the mobile telephone (e.g. the camera) or utilizes a device attached to the mobile telephone. The reader may be used to detect and/or measure the signal and provide data to the user of the mobile telephone application. Alternatively, or in addition, the reader can send the data to a computer data base, such as a cloud-based database, for analysis and then transmission and/or displays of the results on the mobile telephone.

### Interpreting the Results

While the signal may be interpreted as a binary result, e.g., a “yes/no” result, depending on the components used in the assay and the source of the sample, the numerical values produced from an reader, for example, may be plotted against a previously prepared standard to provide the specific amount of the detected analyte (e.g., in ng/ml) in the sample and may be compared against a threshold to produce a result. In some embodiments, the signal may be interpreted as a result including levels, e.g., a “low/medium/high” result.

### Pre-Treatment of Samples Prior to Diagnosis

While samples can be applied directly to a detection device from their source, in some instances pre-treatment of a sample prior to contacting the detection device with the sample can not only increase the sensitivity of an MTM-based detection method but can also increase the spectrum of microbes that can be detected using the detection devices of the invention.

Pre-treatment includes the use of filters or columns to remove large-sized contaminants, and centrifugation to remove smaller-sized contaminants. Alternatively, or in addition, a sample may be incubated with one or more antimicrobial agents, antibiotics, antifungals or antivirals to release target molecules from the organism. In some aspects, the pre-treatment can comprise incubating the sample with at least one or more degradative enzymes. For example, in some aspects, an enzyme such as a protease can be used to cleave cell wall carbohydrates from the microbes, thus allowing detection of carbohydrates that are otherwise not recognized by MTMs.

### Examples—Reactivity with Yeast, Gram-Negative Bacteria, and Gram-Positive Bacteria

### A. Mannan-Spiked in Blood Samples

Human blood samples were obtained (Research Blood Components, Watertown, MA) and treated with either heparin (15.8 units per mL of blood) or EDTA (1.8 mg per mL of blood).

Separate dilutions of mannan (Saccharomyces cerevisiae; Sigma Cat. No. M7504), ranging from 0.5 and 80 ng/mL, in 20 ul samples of heparinized blood or 20 ul samples of EDTA-treated, blood were prepared and LFA was performed.

The LFA platform shown in FIG. 3 was used in the experiments. Details regarding the main device components: Sample Pad—MF1 GE 19 mm (treated with 0.5% Tween-20 and 150 mM NaCl. Conjugate Pad—Ahlstrom Synthetic Conjugate Pads 6614, sprayed with 0.1% Eu-FcMBL and 0.03% Eu-IgY at location 1 and 0.75 uM PEG-Biotin-MBL with 0.2% Tween-20 at location 2. Membrane—CN95 nitrocellulose membrane, coated 0.5 mg/mL polystreptavidin at test line and 0.5 mg/mL anti-IgY at control line (with 2% sucrose and 1% trehalose stabilizers). Absorbent Pad—Ahlstrom 243 wick pad, cut to 15 mm. Housing—plastic cassettes, Custom molded 1384.

The device was constructed using the following steps:
1. laminate card using 60 mm backing card;
2. laminate Membrane 24 mm from the bottom of the backing card;
3. laminate Absorbent Pad from the top of the backing card, ensuring a 2 mm overall with Membrane;
4. measure 3 mm on the end of the membrane and laminate Conjugate Pad;
5. laminate Sample Pad aligned with the bottom edge of the backing card, ensuring 2 mm overlap with the conjugate pad;
6. use kinematic to cut cards to 4.9 mm width strips.

The MTM binding agents were (i) FcMBL (SEQ ID NO:6) labeled with Eu particles and (ii) biotinylated MBL. The capture component was streptavidin. 75 μl of a running buffer (12 mM HEPES, pH, 7.0, 1.2% Tween-20, 0.6 M NaCl, 10 mM CaCl₂, 8 mM glucose) was applied to each Sample Pad 1 min. after the sample (20 ul) was applied. The reaction time was minutes. An Axxin AX-2X-S Fluorescent Reader was used to measure the quantitative output of test line and control line intensities.

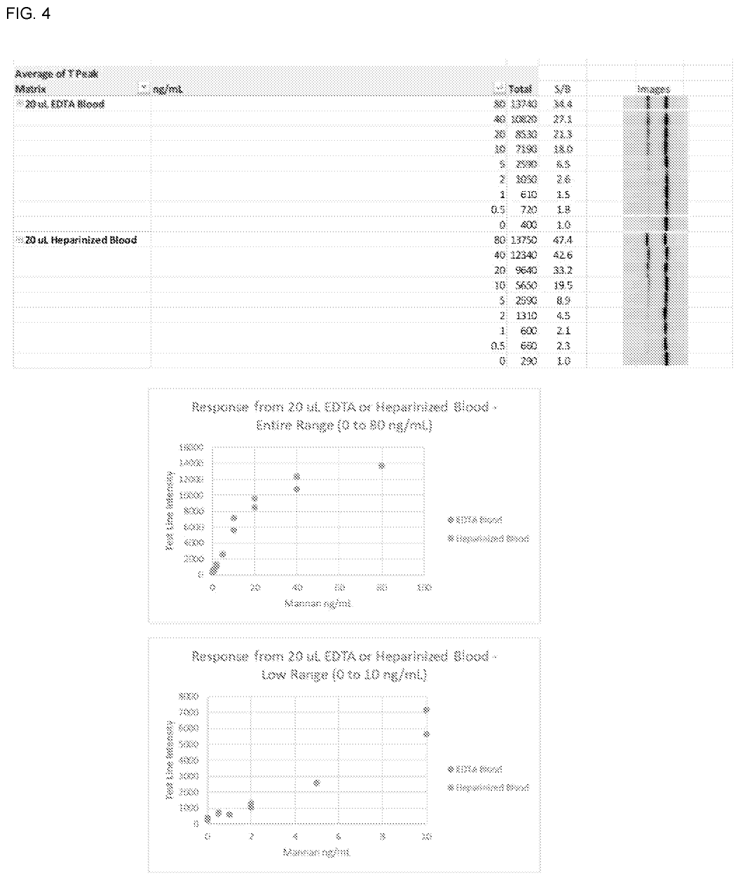

The results shown in FIG. 4 show reactivity to the samples spiked with mannan. Results in both EDTA and heparinized blood indicate sensitivity is in the 2 ng/mL range for mannan (S/B>2). Response profiles were similar in both sample types. This performance was quite similar to PAMPs ELISA assay (data not shown).

### B. LPS (Gram-Negative Bacterial Antigen) Spiked in Blood Samples

Human blood samples were obtained (Research Blood Components, Watertown, MA) and treated with heparin (15.8 units per mL of blood).

Dilutions of lipopolysaccharide (LPS) (E. coli O111:B4; Millipore Sigma), ranging from 625 and 20,000 ng/mL, in 20 ul samples of heparinized blood, were prepared and applied to the LFA of the invention.

The LFA platform described above in A. was used in the experiments. The binding agents were (i) FcMBL labeled with Eu particles and (ii) biotinylated MBL. The capture component was streptavidin. 75 μl of a running buffer (12 mM HEPES, pH, 7.0, 1.2% Tween-20, 0.6 M NaCl, 10 mM CaCl₂) was applied to each Sample Pad 1 min. after the sample (20 ul) was applied. The reaction time was 10-15 minutes. An Axxin AX-2X-S Fluorescent Reader was used to measure the quantitative output of test line and control line intensities.

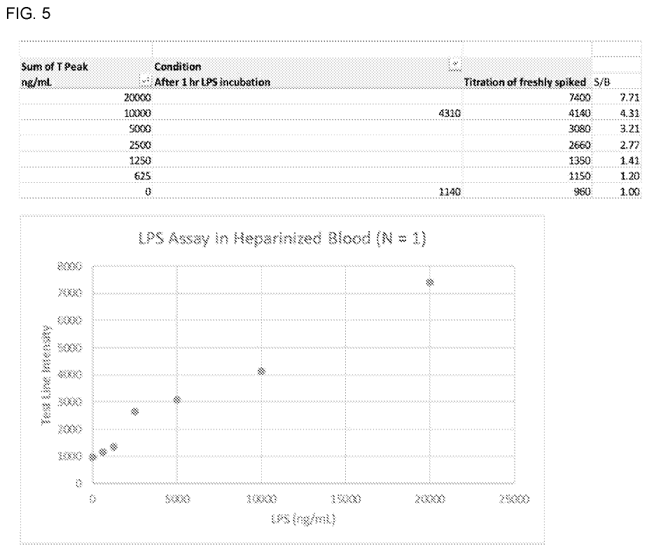

The results shown in FIG. 5 show reactivity at around 2000 ng/ml (in between 1350-2660 ng/mL), which was similar to ELISA assay (data snot shown).

### C. LTA (Gram-Positive Bacterial Antigen) Spiked in Blood Samples

Human blood samples were obtained (Research Blood Components, Watertown, MA) and treated with heparin (15.8 units per mL of blood).

Dilutions of lipoteichoic acid (LTA) (Staphylococcus aureus; Sigma-Aldrich Cat. No. L2515), ranging from 500 and 10,000 ng/mL, in 20 ul samples of heparinized blood were prepared and applied to the LFA of the invention.

T [symbol] The LFA platform described above in A. was used in the experiments. The binding agents were (i) FcMBL labeled with Eu particles and (ii) biotinylated MBL. The capture component was streptavidin. 75 μl of a running buffer (12 mM HEPES, pH, 7.0, 1.2% Tween-20, 0.6 M NaCl, 10 mM CaCl₂) was applied to each Sample Pad 1 min. after the sample (20 ul) was applied. The reaction time was 10-15 minutes. An Axxin AX-2X-S Fluorescent Reader was used to measure the quantitative output of test line and control line intensities.

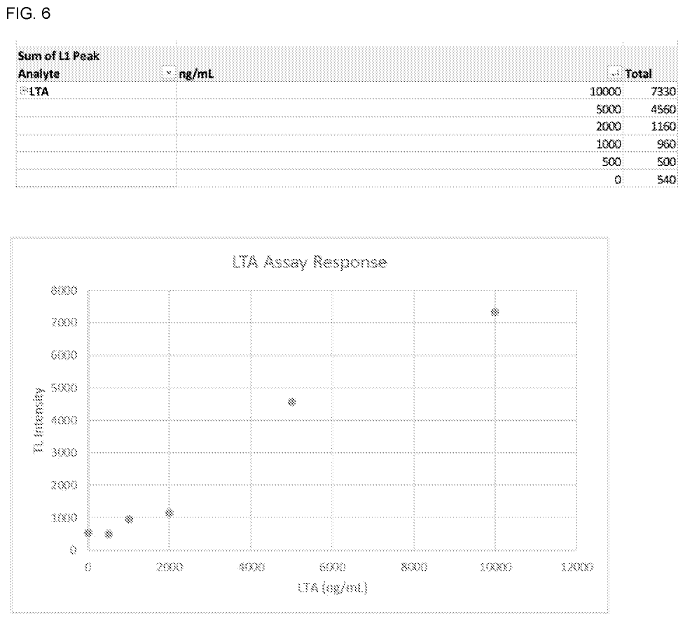

The results shown in FIG. 6 indicate the assay responds down to 100 ng/mL of LTA in heparinized blood.

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the invention and many further aspects thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various aspects and equivalents thereof.

1. A detection device comprising at least one component coated with, or otherwise displaying, one or more microbe-targeting molecules (MTMs),
wherein the one or more MTMs comprise a collectin-based engineered MTM comprising at least one collectin microbe-binding domain and at least one additional domain, wherein the collectin microbe-binding domain comprises the carbohydrate recognition domain (CRD) of a collectin selected from the group consisting of
(i) mannose-binding lectin (MBL),
(ii) surfactant protein A (SP-A),
(iii) surfactant protein D (SP-D),
(iv) collectin liver 1 (CL-L1),
(v) collectin placenta 1 (CL-P1),
(vi) conglutinin collectin of 43 kDa (CL-43),
(vii) collectin of 46 kDa (CL-46),
(viii) collectin kidney 1 (CL-K1),
(ix) conglutinin, and
(x) a sequence variant having at least 85% sequence identity to any one of (i)-(ix), and wherein the at least one additional domain is one or more domains selected from the group consisting of
(xi) a collectin cysteine-rich domain,
(xii) a collectin collagen-like domain,
(xiii) a collectin coiled-coil neck domain,
(xiv) a ficolin short N-terminal domain,
(xv) a ficolin collagen-like domain,
(xvi) a TLR transmembrane helix,
(xvii) a TLR C-terminal cytoplasmic signaling domain,
(xviii) an oligomerization domain,
(xix) a signal domain,
(xx) an anchor domain,
(xxi) a collagen-like domain,
(xxii) a fibrinogen-like domain,
(xxiii) an immunoglobulin domain,
(xxiv) an immunoglobulin-like domain, and
(xxv) a sequence variant having at least 85% sequence identity to any one of (xi)-(xxiv).

2. The detection device of claim 1, wherein the device is a dipstick, a test strip, or a combination of test strips.

3. The detection device of claim 2, wherein the device is a combination of test strips on a solid support.

4. The detection device of claim 3, wherein the device is a lateral flow assay (LFA).

5. The detection device of claim 4, wherein the LFA comprises: (a) a Backing Card, (b) a Sample Pad for receiving a liquid sample, and optionally a running buffer, arranged on a first end of the Backing Card, (c) a Conjugate Pad comprising one or more mobilizable labeled MTMs, (d) a Membrane comprising a detection zone comprising (i) an immobilized first capture component against the labeled MTM in the shape of a first line, and (ii) optionally an immobilized second capture component against a control analyte in the shape of a second line arranged at a distance from the first line, and (e) an Absorbent Pad at a second end of the Backing Card, wherein the Sample Pad, the Conjugate Pad, the Membrane, and the Absorbent Pad are mounted to the Backing Card to permit capillary flow from the Sample Pad into the Conjugate Pad, from Conjugate Pad into the Membrane, and from the Membrane into the Absorbent Pad.

6. The detection device of claim 5, wherein the labeled MTM is labeled with a colored particle, a fluorescent particle, colloidal gold or latex.

7-9. (canceled)

10. The detection device of claim 1, wherein the dipstick or test strips comprise filters, absorbent pads, absorbent paper, fibers, screens, mesh, tubes, hollow fibers, membranes, or any combinations thereof.

11. The detection device of claim 1, wherein the device is a dipstick, a test strip, or a combination of test strips within a housing.

12. The detection device of claim 1, wherein the detection device comprises two or more MTMs having different binding specificities.

13. The detection device of claim 1, wherein the detection device comprises a first MTM and a second MTM, wherein the first and second MTMs have different binding specificities, and wherein the first MTM is affixed to the component in a first predetermined pattern and the second MTM is affixed to the component in a second predetermined pattern.

14. (canceled)

15. The detection device of claim 1, wherein the collectin microbe-binding domain comprises the CRD of MBL or a sequence variant thereof having at least 85% sequence identity to the CRD of MBL.

16. The detection device of claim 1, wherein the CRD of MBL comprises the amino acid sequence of any one of SEQ ID NOs:1, 2, 3, 4, and 5 or a sequence variant thereof having at least 85% sequence identity to any one of SEQ ID NOs:1, 2, 3, 4, and 5.

17. The detection device of claim 1, wherein the at least one additional domain is an immunoglobulin domain.

18. The detection device of claim 17, wherein the immunoglobulin domain comprises the amino acid sequence of SEQ ID NO:9 or a sequence variant thereof having at least 85% sequence identity to SEQ ID NO:9.

19. The detection device of claim 1, comprising at least one collectin-based engineered MTM, wherein the collectin-based engineered MTMs is an FcMBL of SEQ ID NO:6, 7 or 8 or a sequence variant thereof having at least 85% sequence identity to SEQ ID NO:6, 7 or 8.

20-28. (canceled)

29. A method of detecting a microbe in a sample, comprising contacting a sample suspected of containing a microbe with a detection device of claim 1 under conditions permitting binding of a microbe or a microbial component by MTMs displayed by the at least one component of the detection device, thereby detecting a microbe in a sample.

30. A method of detecting a microbial infection in a subject, comprising contacting a biological sample of a subject suspected of having a microbial infection with a detection device of claim 1 under conditions permitting binding of microbes or microbial components by MTMs displayed by the at least one component of the detection device, thereby detecting a microbial infection in the subject.

31. The method of claim 29, wherein the sample is a biological sample or water.

32. (canceled)

33. A method of detecting a microbe in a sample, comprising the steps of (a) applying a sample to the Sample Pad of the device according to claim 5 under conditions permitting flow of the sample from the Sample Pad through the Conjugate Pad to the Membrane, (b) detecting the presence and/or amount of the labeled MTMs in the detection zone, and (c) optionally detecting the presence or amount of the control analyte in the detection zone, wherein the presence or absence or the detected amount of the labeled MTMs in the detection zone is indicative for the absence or presence or the amount of the microbe in the sample.

34. A method of detecting a microbial infection in a subject, comprising contacting a biological sample of a subject suspected of having a microbial infection with a detection device of claim 1 under conditions permitting binding of microbes or microbial components by MTMs displayed by the at least one component of the detection device, thereby detecting a microbial infection in the subject.
